(19) United States
(12) Patent Application Publication
(10) Pub. No.: US 2023/0124374 A1
(43) Pub. Date: Apr. 20, 2023

(54) COMPOSITIONS AND METHODS FOR EYE HEALTH COMPRISING VERY LONG CHAIN FATTY ACIDS

(71) Applicant: BAUSCH + LOMB IRELAND LIMITED, Dublin (IE)

(72) Inventors: Daniel J. STEIN, Bridgewater, NJ (US); Melinda DIVITO, Bridgewater, NJ (US); Lester HOSTEN, Bridgewater, NJ (US)

(73) Assignee: BAUSCH + LOMB IRELAND LIMITED, Dublin (IE)

(21) Appl. No.: 17/905,930

(22) PCT Filed: Mar. 11, 2021
(86) PCT No.: PCT/IB2021/052007

§ 371 Date: Sep. 8, 2022

Related U.S. Application Data
(60) Provisional application No. 63/045,609, filed on Jun. 29, 2020.
(60) Provisional application No. 62/988,386, filed on Mar. 11, 2020.

(51) Int. Cl. A61K 31/202; A61K 31/375; A61K 31/355; A61K 33/30; A61K 33/34; A61K 31/047; A61K 31/51; A61K 31/525; A61K 31/455; A61K 31/197; A61K 31/519; A61K 31/714; A61P 27/02

(52) U.S. Cl. CPC A61K 31/202; A61K 31/375; A61K 31/355; A61K 33/30; A61K 33/34; A61K 31/047; A61K 31/51; A61K 31/525; A61K 31/455; A61K 31/197; A61K 31/519; A61K 31/714; A61P 27/02

(57) ABSTRACT

The present disclosure describes compositions comprising at least one very long chain fatty acid chosen from C₂₆₊ acids (5n-3), prodrugs thereof, and pharmaceutically acceptable salts of any of the foregoing. The compositions are useful in methods for treating conditions associated with a loss of visual acuity including, for example, age-related macular degeneration.

Compositions and methods for treating and/or preventing at least one disease, disorder, and/or condition associated with a loss of visual acuity including, for example, age-related macular degeneration, are disclosed herein. In some embodiments, the compositions disclosed herein comprise at least one very long chain fatty acid chosen from C₂₆₊ acids (5n-3), prodrugs thereof, and pharmaceutically acceptable salts of any of the foregoing.

Age-related macular degeneration (AMD) is a leading cause of severe visual acuity loss in the United States and Western Europe in persons aged 55 years or older. An estimated 1.75 million individuals in the United States have advanced AMD, which accounts for most cases of severe vision loss. An additional 7.3 million persons have early AMD, which is usually associated with little or no vision loss but increases the risk of developing AMD. It is associated with a collection of clinically recognizable ocular findings that can lead to blindness. These findings include drusen, retinal pigment epithelial (RPE) disturbance, including pigment clumping and/or dropout, RPE detachment, geographic atrophy, subretinal neovascularization and disciform scar. Not all these manifestations are needed for AMD to be considered present.

Disclosed herein are compositions for eye health, for example, for preventing or treating macular degeneration. In some embodiments, the compositions disclosed herein are nutritional or dietary supplement compositions. In some embodiments, the compositions are pharmaceutical compositions. In some embodiments, the compositions disclosed herein may strengthen and/or promote retinal health, for example through stabilization and/or treatment of visual acuity loss in people with particular ocular diseases, disorders, and/or conditions. In some embodiments, the disclosure relates to antioxidant nutritional supplements comprising a potent vitamin B complex. In some embodiments, administration of the compositions may decrease visual acuity loss, for example by reducing the risk of developing late stage or advanced age-related macular degeneration in patients.

Also disclosed herein are methods for treatment and/or prevention of at least one disease, disorder, and/or condition associated with loss of visual acuity, the method comprising administering to a subject in need thereof a composition as disclosed herein. In some embodiments, the at least one disease, disorder, and/or condition associated with a loss of visual acuity is chosen from age-related macular degeneration (AMD), atrophy of retinal pigmented epithelium (RPE), atrophy of at least one photoreceptor, drusen, drusenoid pigment epithelial detachment (PED), diabetic retinopathy, cataracts, retinitis pigmentosa, glaucoma, choroidal neovascularization, retinal degeneration, and oxygen-induced retinopathy.

Also disclosed herein are methods of regressing drusen and/or drusenoid pigment epithelial detachment (PED), methods for treating and/or preventing atrophy of retinal pigmented epithelium (RPE) and/or at least one photoreceptor, and methods for treating and/or preventing vision loss and/or improving acuity. The methods comprise administering to a subject in need thereof a composition as disclosed herein.

### FIGURES

FIGS. 1A and 1B show wild-type (WT) mice single-dose bioavailability studies: (1A) Serum absorption kinetics of C₃₂ acid (6n-3) (“32:6n-3”) after single-dose gavage feeding of 6 mg/mouse (n=4 mice/time point) and (1B) retina and RPE uptake of 32:6 n-3 after single-dose gavage feeding. No VLCPUFAs were detected in liver, brain, or RBC membranes at any time point. Data are presented as mean±SEM (***P<0.005; **P<0.01; *P<0.05).

FIGS. 2A and 2B show WT mice repeated-dose bioavailability studies: (2A) Accumulation of 32:6 n-3 in mouse retina and RPE after 15 d of gavage feeding (n=6 mice/group) and (2B) Bioaccumulation of 32:6n-3 in serum, RBC, and liver after 15 d of gavage feeding. No VLCPUFAs were ever detectable in the brain. Data are presented as mean±SEM (***P<0.005; **P<0.01; N.S.: not significant; N.D.: not detectable).

FIGS. 3A and 3B show phospholipid analysis of wild type (WT) mice after 15 days of gavage feeding of VLCPUFA 32:6n-3 (2 mg/day): (3A) Retina and (3B) RPE. Data presented as mean±SEM (n=8 mice/group) (p values: *p<0.05; N.S.: not significant; N.D.: not detectable).

FIGS. 4A and 4B show long-term uptake of 32:6n-3 (2 mg/d per mouse) in Elov14 rod-cone conditional KO (E4cKO) mice: (4A) Accumulation of 32:6 n-3 in E4cKO mouse retinas and RPE in comparison to age-matched WT mice after 15 d of gavage feeding (n=6 mice/group) and (4B) Accumulation of 32:6n-3 in liver and RBCs in E4cKO mice in comparison to age-matched WT mice after 15 d of gavage feeding. Data are presented as mean±SEM (**P<0.01; N.S.: not significant; N.D.: not detectable).

Definitions of certain terms as used in this application are provided below. Unless defined otherwise, all technical and scientific terms used herein have the normal and common meaning that would be commonly understood by one of ordinary skill in the art to which this disclosure belongs.

As used herein, “a,” “an,” and “the” refer to one or more (i.e., to at least one) of the grammatical object of the article.

As used herein, the terms “Very Long Chain Fatty Acids” (“VLCFAs”) and “Very Long Chain Polyunsaturated Fatty Acids” (“VLCPUFAs”) are interchangeable and refer to fatty acids with a chain length greater than 26 carbons (such as 26 to 60 carbons, such as to 26 to 40 carbons, or 26 to 34 carbons), and 3 to 6 double bonds. The proximal carboxylic region of these molecules is comprised of 12 to 20 saturated carbon chains, and the distal region (methyl group end) contains 4 or more conjugated methylene cis double bonds.

As used herein “C₂₆₊ acids (5n-3)” (used interchangeably herein with the term “C₂₆₊ eicosapentanoic acids (5n-3)”) are very long chain fatty acids with a chain length of at least 26 carbons, such as 26 to 36 carbons, such as 26, 28, 30, 32, or 34 carbons, and 5 double bonds. C₂₆₊ acids (5n-3) may be in the form of an acid, a prodrug thereof, or a pharmaceutically acceptable salt any of the foregoing.

As used herein, and “C₂₆₊ acids (6n-3)” (used interchangeably herein with the term “C₂₆₊ docosahexanoic acids (6n-3)”) are very long chain fatty acids with a chain length of at least 26 carbons, such as 26 to 36 carbons, and 6 double bonds. C₂₆₊ acids (6n-3) may be in the form of an acid, a prodrug thereof, or a pharmaceutically acceptable salt any of the foregoing.

As used herein, administration of a “daily” amount of a recited element or composition refers to the total amount that is administered in one day but does not limit the frequency of administration per day. The daily amount administered to a patient can be administered once or multiple times in a day, such as twice daily or three times daily (wherein each of multiple administrations comprises administering some amount of a recited element or composition that is less than the “daily” amount, given that the “daily” amount refers to the total amount administered in one day). Each administration of a recited element or composition can consist of administering the recited element or composition in the form of a single dosage (e.g., such as a single tablet or a single capsule) or in the form of multiple dosages (e.g., such as multiple (i.e., two or more) tablets and/or capsules).

As used herein, the terms “treat” and “treatment” include medical management of a disease, disorder, and/or condition of a subject as would be understood by a person of ordinary skill in the art (see, e.g., Stedman's Medical Dictionary). In general, an appropriate dose and treatment regimen provide at least one of the compositions of the present disclosure sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or lessen an undesired physiological change or disorder, or to prevent or slow or lessen the expansion or severity of such disorder. As discussed herein, beneficial or desired clinical results from treating a subject include, but are not limited to, abatement, lessening, or alleviation of symptoms that result from or are associated with the disease, condition, and/or disorder to be treated; decreased occurrence of symptoms; improved quality of life; longer symptom-free status (i.e., decreasing the likelihood or the propensity that a subject will present symptoms on the basis of which a diagnosis of a disease is made); diminishment of extent of disease, disorder, and/or condition; stabilized (i.e., not worsening) state of disease, disorder, and/or condition; delay or slowing of progression of a disease, disorder, and/or condition; amelioration or palliation of the state of a disease, disorder, and/or condition; and remission (whether partial or total), whether detectable or undetectable; and/or overall survival.

As used herein, “prevention” of or “preventing” a disorder or condition refers to reduction of or reducing the occurrence of the disorder or condition in a treated sample relative to an untreated control sample, and includes delaying onset, progression, or reduction of severity of one or more symptoms of the disorder or condition relative to the untreated control sample.

As used herein, the term “pharmaceutically acceptable salt” refers to a salt form of a compound wherein the salt is nontoxic and includes such salts derived from suitable inorganic and organic acids and bases. Pharmaceutically acceptable salts are well known in the art. For example, S. M. Berge, et al. describes pharmaceutically acceptable salts in detail in J. Pharmaceutical Sciences, 1977, 66, 1-19. Non-limiting examples of suitable pharmaceutically acceptable acid addition salts include chlorides, bromides, sulfates, nitrates, phosphates, sulfonates, methane sulfonates, formates, tartrates, maleates, succinates, malonates, citrates, benzoates, salicylates, and ascorbates. Non-limiting examples of suitable pharmaceutically acceptable base addition salts include sodium, potassium, lithium, ammonium (substituted and unsubstituted), calcium, magnesium, iron, zinc, copper, manganese, and aluminum salts. Non-limiting examples of pharmaceutically acceptable salts include pharmaceutically acceptable salts derived from appropriate bases and include alkali metal, alkaline earth metal, ammonium, and N⁺(C₁₋₄alkyl)₄ salts. This disclosure also envisions the quaternization of any basic nitrogen-containing groups of the compounds disclosed herein. Pharmaceutically acceptable salts may, for example, be obtained using standard procedures well known in the field of pharmaceuticals. One of ordinary skill in the art will recognize that the stability and other properties of different pharmaceutically acceptable salts of the recited components herein may differ and will consider these differences when selecting suitable pharmaceutically acceptable salt(s).

The terms “patient,” “subject,” “individual,” and the like, as used herein, are interchangeable and refer to any animal, which may be a human or a non-human animal.

As used herein, “age-related macular degeneration” or “AMD” includes all forms of macular degeneration, including “wet” (exudative) and “dry” (atrophic) forms.

The term “prodrug” includes compounds that may be converted (e.g., under physiological conditions or by solvolysis) to a biologically active compound. Thus, the term “prodrug” includes metabolic precursors of compounds that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release active compound(s) as described herein in vivo when such prodrug is administered to a subject. Non-limiting examples of prodrugs include ester and amide derivatives of hydroxy, carboxy, mercapto and amino functional groups in the compounds described herein.

The term “nutritional composition,” as used herein, includes a food product intended for human consumption.

As will be understood by one of ordinary skill in the art, when disclosed herein, each range includes all possible subranges as well as individual numerical values within that range. For example, a range of “1.0 to 5.0” includes and would be understood to specifically disclose subranges such as “1.0 to 3.0,” “1.5 to 3.7,” “2.1 to 4.3, etc., as well as all individual numbers within the disclosed range, for example, 1.0, 1.1, 1.2, 1.3, etc.

Disclosed herein are compositions comprising at least one very long chain fatty acid chosen from C₂₆₊ acids (5n-3), prodrugs thereof, and pharmaceutically acceptable salts of any of the foregoing. In some embodiments, the at least one very long chain fatty acid chosen from C₂₆₊ acids (5n-3) is a C₂₆ acid (5n-3), C₂₈ acid (5n-3), C₃₀ acid (5n-3), C₃₂ acid (5n-3), C₃₄ acid (5n-3), or a mixture of any one or more thereof.

In some embodiments, the composition further comprises at least one additional component chosen from at least one very long chain fatty acid chosen from C₂₆₊ acids (6n-3), prodrugs thereof, and pharmaceutically acceptable salts of any of the foregoing; docosahexanoic acid, prodrugs thereof, and/or pharmaceutically acceptable salts of any of the foregoing; eicosapentanoic acid, prodrugs thereof, and/or pharmaceutically acceptable salts of any of the foregoing.

In some embodiments, the composition further comprises at least one additional component chosen from at least one very long chain fatty acid chosen from C₂₆₊ acids (6n-3), prodrugs thereof, and pharmaceutically acceptable salts of any of the foregoing; docosahexanoic acid, prodrugs thereof, and/or pharmaceutically acceptable salts of any of the foregoing; eicosapentanoic acid, prodrugs thereof, and/or pharmaceutically acceptable salts of any of the foregoing; vitamin C, prodrugs thereof, and/or pharmaceutically acceptable salts of any of the foregoing; vitamin E, prodrugs thereof, and/or pharmaceutically acceptable salts of any of the foregoing; zinc, prodrugs thereof, and/or pharmaceutically acceptable salts of any of the foregoing; copper, prodrugs thereof, and/or pharmaceutically acceptable salts of any of the foregoing; lutein, prodrugs thereof, and/or pharmaceutically acceptable salts of any of the foregoing; and zeaxanthin, prodrugs thereof, and/or pharmaceutically acceptable salts of any of the foregoing.

In some embodiments, the composition comprises 0.1 mg to 1000.0 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the composition comprises 1.0 mg to 900.0 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the composition comprises 100.0 mg to 500.0 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the composition comprises 200.0 mg of at least one C₂₆₊ acid (5n-3) or an equivalent amount of prodrugs, pharmaceutically acceptable salts, and/or pharmaceutically acceptable salts of prodrugs of C₂₆₊ acids (5n-3). In some embodiments, the composition comprises 40.0 mg of at least one very long chain fatty acid chosen from C₂₆₊ acids (5n-3). In some embodiments, the composition comprises 40.0 mg of C₃₀ acid (5n-3). In some embodiments, the composition comprises 40.0 mg of C₃₄ acid (5n-3). In some embodiments, the at least one very long chain fatty acid chosen from C₂₆₊ acids (5n-3) is a C₂₆ acid (5n-3), C₂₈ acid (5n-3), C₃₀ acid (5n-3), C₃₂ acid (5n-3), C₃₄ acid (5n-3), or a mixture of any one or more thereof.

In some embodiments, the composition comprises about 1 mg to about 1000 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the composition comprises about 10 mg to about 250 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the composition comprises about 200 mg to about 250 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the composition comprises about 200 mg of at least one C₂₆₊ acid (5n-3) or an equivalent amount of prodrugs, pharmaceutically acceptable salts, and/or pharmaceutically acceptable salts of prodrugs of C₂₆₊ acids (5n-3). In some embodiments, the at least one very long chain fatty acid chosen from C₂₆₊ acids (5n-3) is a C₂₆ acid (5n-3), C₂₈ acid (5n-3), C₃₀ acid (5n-3), C₃₂ acid (5n-3), C₃₄ acid (5n-3), or a mixture of any one or more thereof.

In some embodiments, the composition comprises about 1 mg to about 100 mg of at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the composition comprises about 1 mg to about 50 mg of the at least one very long chain fatty acid, such as about 10 mg to about 50 mg, about 10 mg to about 40 mg, about 20 mg to about 40 mg, about 20 mg to about 30 mg, about 30 mg to about 40 mg, of at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the composition comprises about 50 mg to about 100 mg, such about 50 mg to about 75 mg, about 60 mg to about 90 mg, about 55 mg to about 85 mg, about 70 mg to about 95 mg, or about 65 mg to about 80 mg of at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the at least one very long chain fatty acid chosen from C₂₆₊ acids (5n-3) is a C₂₆ acid (5n-3), C₂₈ acid (5n-3), C₃₀ acid (5n-3), C₃₂ acid (5n-3), C₃₄ acid (5n-3), or a mixture of any one or more thereof.

In some embodiments, the composition comprises about 100 mg to about 1000 mg, such as about 150 mg to about 900 mg, about 250 mg to about 800 mg, about 350 mg to about 700 mg, about 450 mg to about 600 mg, or about 500 mg to about 550 mg, of at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the at least one very long chain fatty acid chosen from C₂₆₊ acids (5n-3) is a C₂₆ acid (5n-3), C₂₈ acid (5n-3), C₃₀ acid (5n-3), C₃₂ acid (5n-3), C₃₄ acid (5n-3), or a mixture of any one or more thereof.

In some embodiments, the composition comprises 0.1 mg, 0.25 mg, 0.5 mg, 0.6 mg, 0.75 mg, 0.9 mg, 1 mg, 2.5 mg, 3 mg, 5 mg, 7.5 mg, 10 mg, 15 mg, 20, mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, or 1000 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the at least one very long chain fatty acid chosen from C₂₆₊ acids (5n-3) is a C₂₆ acid (5n-3), C₂₈ acid (5n-3), C₃₀ acid (5n-3), C₃₂ acid (5n-3), C₃₄ acid (5n-3), or a mixture of any one or more thereof.

In some embodiments, the composition comprises more than zero but less than 3 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3). In some embodiments, the at least one very long chain fatty acid chosen from C₂₆₊ acids (5n-3) is a C₂₆ acid (5n-3), C₂₈ acid (5n-3), C₃₀ acid (5n-3), C₃₂ acid (5n-3), C₃₄ acid (5n-3), or a mixture of any one or more thereof.

In some embodiments, the composition further comprises 1.0 mg to 1000.0 mg of at least one additional component chosen from at least one very long chain fatty acid chosen from C₂₆₊ acids (6n-3), prodrugs thereof, and pharmaceutically acceptable salts of any of the foregoing. In some embodiments, the composition further comprises 150.0 mg to 250.0 mg of docosahexanoic acid or an equivalent amount of a prodrug, a pharmaceutically acceptable salt, and/or a pharmaceutically acceptable salt of a prodrug of docosahexanoic acid. In some embodiments, the composition further comprises 35.0 mg to 45.0 mg of eicosapentanoic acid or an equivalent amount of a prodrug, a pharmaceutically acceptable salt, and/or a pharmaceutically acceptable salt of a prodrug of eicosapentanoic acid.

In some embodiments, the composition further comprises 350.0 mg to 800.0 mg of vitamin C or an equivalent amount of a prodrug, a pharmaceutically acceptable salt, and/or a pharmaceutically acceptable salt of a prodrug of vitamin C. In some embodiments, the composition further comprises 50.0 mg to 550.0 mg of vitamin E or an equivalent amount of a prodrug, a pharmaceutically acceptable salt, and/or a pharmaceutically acceptable salt of a prodrug of vitamin E. In some embodiments, the composition further comprises 20.0 mg to 90.0 mg of zinc or an equivalent amount of a prodrug, a pharmaceutically acceptable form, and/or a pharmaceutically acceptable salt of a prodrug of zinc. In some embodiments, the composition further comprises 1.5 mg to 2.5 mg of copper or an equivalent amount of a prodrug, a pharmaceutically acceptable form, and/or a pharmaceutically acceptable salt of a prodrug of copper. In some embodiments, the composition further comprises 5.0 mg to 50.0 mg of lutein or an equivalent amount of a prodrug, a pharmaceutically acceptable salt, and/or a pharmaceutically acceptable salt of a prodrug of lutein. In some embodiments, the composition further comprises 1.0 mg to 20.0 mg of zeaxanthin or an equivalent amount of a prodrug, a pharmaceutically acceptable salt, and/or a pharmaceutically acceptable salt of a prodrug of zeaxanthin.

In some embodiments, the composition comprises:

20.0 mg at least one C₂₆₋₃₄ acid (5n-3);

200.0 mg docosahexanoic acid; and

40.0 mg eicosapentanoic acid.

In some embodiments, the composition comprises:

30.0 mg at least one C₂₆₋₃₄ acid (5n-3);

200.0 mg docosahexanoic acid; and

40.0 mg eicosapentanoic acid.

In some embodiments, the composition comprises:

40.0 mg at least one C₂₆₋₃₄ acid (5n-3);

200.0 mg docosahexanoic acid; and

40.0 mg eicosapentanoic acid.

In some embodiments, the composition comprises:

20.0 mg to 40.0 mg at least one C₂₆₋₃₄ acid (5n-3);

450.0 mg vitamin C, optionally in the form of ascorbic acid;

100.0 mg vitamin E, optionally in the form of dl-alpha tocopheryl acetate; and

80.0 mg zinc.

In some embodiments, the composition comprises:

40.0 mg at least one C₂₆₋₃₄ acid (5n-3);

500.0 mg vitamin C, optionally in the form of ascorbic acid;

180.0 mg vitamin E, optionally in the form of dl-alpha tocopheryl acetate;

80.0 mg zinc, optionally in the form of zinc oxide;

2.0 mg copper, optionally in the form of copper oxide;

200.0 mg docosahexanoic acid; and

40.0 mg eicosapentanoic acid.

In some embodiments, the composition comprises:

40.0 mg at least one C₂₆₋₃₄ acid (5n-3);

500.0 mg vitamin C, optionally in the form of ascorbic acid;

180.0 mg vitamin E, optionally in the form of dl-alpha tocopheryl acetate;

80.0 mg zinc, optionally in the form of zinc oxide;

2.0 mg copper, optionally in the form of copper oxide;

optionally up to 200.0 mg at least one C₂₆₋₃₄ acid (6n-3);

200.0 mg docosahexanoic acid; and

40.0 mg eicosapentanoic acid.

In some embodiments, the composition further comprises 10.0 mg lutein and 2.0 mg zeaxanthin.

In some embodiments, the composition further comprises about 150 mg to about 250 mg of docosahexanoic acid or an equivalent amount of a prodrug, a pharmaceutically acceptable salt, and/or a pharmaceutically acceptable salt of a prodrug of docosahexanoic acid.

In some embodiments, the composition further comprises about 35 mg to about 45 mg of eicosapentanoic acid or an equivalent amount of a prodrug, a pharmaceutically acceptable salt, and/or a pharmaceutically acceptable salt of a prodrug of eicosapentanoic acid.

In some embodiments, the composition further comprises about 350 mg to about 800 mg of vitamin C or an equivalent amount of a prodrug, a pharmaceutically acceptable salt, and/or a pharmaceutically acceptable salt of a prodrug of vitamin C.

In some embodiments, the composition further comprises about 50 mg to about 550 mg of vitamin E or an equivalent amount of a prodrug, a pharmaceutically acceptable salt, and/or a pharmaceutically acceptable salt of a prodrug of vitamin E.

In some embodiments, the composition further comprises about 20 mg to about 90 mg of zinc or an equivalent amount of a prodrug, a pharmaceutically acceptable form, and/or a pharmaceutically acceptable salt of a prodrug of zinc.

In some embodiments, the composition further comprises about 1.5 mg to about 2.5 mg of copper or an equivalent amount of a prodrug, a pharmaceutically acceptable form, and/or a pharmaceutically acceptable salt of a prodrug of copper.

In some embodiments, the composition further comprises about 5 mg to about 50 mg of lutein or an equivalent amount of a prodrug, a pharmaceutically acceptable salt, and/or a pharmaceutically acceptable salt of a prodrug of lutein.

In some embodiments, the composition further comprises about 1 mg to about 20 mg, such as about 1.5 mg to about 2.5 mg, of zeaxanthin or an equivalent amount of a prodrug, a pharmaceutically acceptable salt, and/or a pharmaceutically acceptable salt of a prodrug of zeaxanthin.

In some embodiments, the composition further comprises about 1 mg to about 1000 mg of the at least one very long chain fatty acid chosen from C₂₆₊ acids (6n-3) or an equivalent amount of a prodrug, pharmaceutically acceptable salt, and/or pharmaceutically acceptable salt of a prodrug of at least one very long chain fatty acid chosen from C₂₆₊ acids (6n-3).

In some embodiments, the composition comprises:

about 20 mg at least one C₂₆₋₃₄ acid (5n-3);

about 200 mg docosahexanoic acid; and

about 40 mg eicosapentanoic acid.

In some embodiments, the composition comprises:

about 30 mg at least one C₂₆₋₃₄ acid (5n-3);

about 200 mg docosahexanoic acid; and

about 40 mg eicosapentanoic acid.

In some embodiments, the composition comprises:

about 40 mg at least one C₂₆₋₃₄ acid (5n-3);

about 200 mg docosahexanoic acid; and

about 40 mg eicosapentanoic acid.

In some embodiments, the composition comprises:

about 500 mg vitamin C, optionally in the form of ascorbic acid;

about 180 mg vitamin E, optionally in the form of dl-alpha tocopheryl acetate;

about 80 mg zinc, optionally in the form of zinc oxide;

about 2 mg copper, optionally in the form of copper oxide;

about 200 mg docosahexanoic acid;

about 40 mg at least one C₂₆₋₃₄ acid (5n-3); and

about 40 mg eicosapentanoic acid.

In some embodiments, the composition comprises:

about 500 mg vitamin C, optionally in the form of ascorbic acid;

about 180 mg vitamin E, optionally in the form of dl-alpha tocopheryl acetate;

about 80 mg zinc, optionally in the form of zinc oxide;

about 2 mg copper, optionally in the form of copper oxide;

about 200 mg docosahexanoic acid;

about 40 mg at least one C₂₆₋₃₄ acid (5n-3); and

about 40 mg eicosapentanoic acid.

In some embodiments, the composition comprises:

about 20 mg to about 40 mg at least one C₂₆₋₃₄ acid (5n-3);

about 450 mg vitamin C, optionally in the form of ascorbic acid;

about 100 mg vitamin E, optionally in the form of dl-alpha tocopheryl acetate; and

about 80 mg zinc, optionally in the form of zinc oxide.

In some embodiments, the compositions further comprises about 10 mg lutein and about 2 mg zeaxanthin.

In some embodiments, the composition comprises at least one C₂₆₋₃₄ acid (5n-3), which can be C₂₆ acid (5n-3) or C₂₈ acid (5n-3) (“EPA (28C)”, below) and/or C₃₀ acid (5n-3) (“EPA (30C)”, below), which are shown in Scheme 1 below:

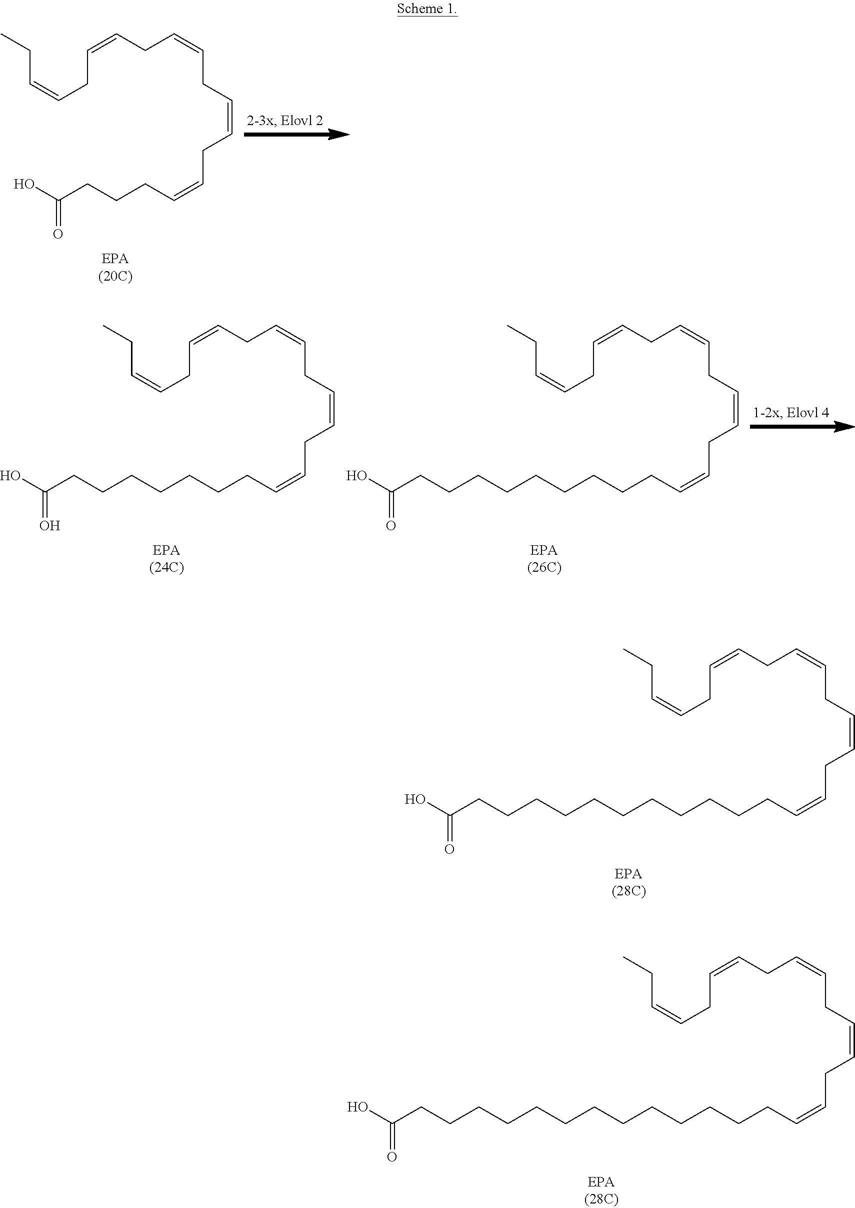 [SMILES: CC/C=C\C/C=C\C/C=C\C/C=C\C/C=C\CCCC(=O)O ; CC/C=C\C/C=C\C/C=C\C/C=C\C/C=C\CCCCCCCC(=O)O ; CC/C=C\C/C=C\C/C=C\C/C=C\C/C=C\CCCCCCCCCC(=O)O ; CC/C=C\C/C=C\C/C=C\C/C=C\C/C=C\CCCCCCCCCCCC(=O)O ; CC/C=C\C/C=C\C/C=C\C/C=C\C/C=C\CCCCCCCCCCCCCC(=O)O]

Additional information can be found, for example, in Do et al., PNAS, 116(48), 24317-24325 (2019), which is incorporated herein by reference in its entirety.

In some embodiments, the composition comprises 0.1 mg to 250 mg of the at least one C₂₆₊ acid (5n-3) or an equivalent amount of prodrugs, pharmaceutically acceptable salts, and/or pharmaceutically acceptable salts of prodrugs of C₂₆₊ acids (5n-3); 150.0 mg to 250.0 mg of docosahexanoic acid or an equivalent amount of a prodrug, a pharmaceutically acceptable salt, and/or a pharmaceutically acceptable salt of a prodrug of docosahexanoic acid; and 35.0 mg to 45.0 mg of eicosapentanoic acid or an equivalent amount of a prodrug, a pharmaceutically acceptable salt, and/or a pharmaceutically acceptable salt of a prodrug of eicosapentanoic acid.

In some embodiments, the composition comprises 0.2 mg to 200 mg at least one C₂₆₊ acid (5n-3); 200.0 mg docosahexanoic acid; and 40.0 mg eicosapentanoic acid.

In some embodiments, the compositions disclosed herein further comprises lutein, a prodrug thereof, and/or a pharmaceutically acceptable salt of lutein. In some embodiments, the lutein, a prodrug thereof, and/or a pharmaceutically acceptable salt of lutein is R′,R′-lutein. Lutein is a carotenoid. In some embodiments, the composition comprises 5.0 mg to 50.0 mg, such as 5.0 mg to 15.0 mg, of lutein or an equivalent amount of a prodrug, a pharmaceutically acceptable salt, and/or a pharmaceutically acceptable salt of a prodrug of lutein. In some embodiments, the composition comprises 5.0 mg to 50.0 mg, such as 6.0 mg to 40.0 mg, 7.0 mg to 30.0 mg, 8.0 mg to 20.0 mg, 9.0 mg to 11.0 mg, 20.0 mg to 30.0 mg, 30.0 mg to 40.0 mg, 40.0 mg to 50.0 mg, 5.0 mg to 15.0 mg, 6.0 mg to 14.0 mg, 7.0 mg to 13.0 mg, 8.0 mg to 12.0 mg, 9.0 mg to 11.0 mg, 10.0 mg, 20.0 mg, 30.0 mg, 40.0 mg, or 50.0 mg of lutein or an equivalent amount of a prodrug, a pharmaceutically acceptable salt, and/or a pharmaceutically acceptable salt of a prodrug of lutein. In some embodiments, the composition comprises 10.0 mg of lutein.

In some embodiments, the compositions disclosed herein further comprises zeaxanthin, a prodrug thereof, and/or a pharmaceutically acceptable salt of zeaxanthin. In some embodiments, the zeaxanthin, a prodrug thereof, and/or a pharmaceutically acceptable salt of zeaxanthin is R′,R′-zeaxanthin. Zeaxanthin is a carotenoid. In some embodiments, the composition comprises 0.01 mg to 40.0 mg, such as 1.5 mg to 2.5 mg, of zeaxanthin or an equivalent amount of a prodrug, a pharmaceutically acceptable salt, and/or a pharmaceutically acceptable salt of a prodrug of zeaxanthin. In some embodiments, the composition comprises 0.04 mg to 40.0 mg, such as 1.0 mg to 30.0 mg, 3.0 mg to 25.0 mg, 5.0 mg to 20.0 mg, 10.0 mg to 20.0 mg, 1.0 mg to 15.0 mg, 3.0 mg to 12.0 mg, 5.0 mg to 20.0 mg, 7.0 mg to 19.0 mg, 8.0 mg to 15.0 mg, 1.5 mg to 2.5 mg, 1.5 mg to 2.0 mg, 2.0 mg to 2.5 mg, or 2.0 mg, of zeaxanthin or an equivalent amount of a prodrug, a pharmaceutically acceptable salt, and/or a pharmaceutically acceptable salt of a prodrug of zeaxanthin. In some embodiments, the composition comprises 2.0 mg of zeaxanthin.

In some embodiments, the compositions disclosed herein comprise about 150 mg to about 250 mg of the at least one C₂₆₊ acid (6n-3) or an equivalent amount of prodrugs, pharmaceutically acceptable salts, and/or pharmaceutically acceptable salts of prodrugs of C₂₆₊ acids (6n-3); about 150 mg to about 250 mg of docosahexanoic acid or an equivalent amount of a prodrug, a pharmaceutically acceptable salt, and/or a pharmaceutically acceptable salt of a prodrug of docosahexanoic acid; about 35 mg to about 45 mg of at least one C₂₆₊ acid (5n-3) or an equivalent amount of prodrugs, pharmaceutically acceptable salts, and/or pharmaceutically acceptable salts of prodrugs of C₂₆₊ acids (5n-3); and about 35 mg to about 45 mg of eicosapentanoic acid or an equivalent amount of a prodrug, a pharmaceutically acceptable salt, and/or a pharmaceutically acceptable salt of a prodrug of eicosapentanoic acid.

In some embodiments, the composition comprises about 20 mg C₃₂ acid (5n-3); about 200 mg docosahexanoic acid; and about 40 mg eicosapentanoic acid.

In some embodiments, the compositions disclosed herein further comprises lutein, a prodrug thereof, and/or a pharmaceutically acceptable salt of lutein. In some embodiments, the lutein, a prodrug thereof, and/or a pharmaceutically acceptable salt of lutein is R′,R′-lutein. Lutein is a carotenoid. In some embodiments, the composition comprises about 5 mg to about 50 mg, such as about 5 mg to about 15 mg, of lutein or an equivalent amount of a prodrug, a pharmaceutically acceptable salt, and/or a pharmaceutically acceptable salt of a prodrug of lutein. In some embodiments, the composition comprises about 5 mg to about 50 mg, such as about 5 mg to about 15 mg, about 6 mg to about 40 mg, about 6 mg to about 14 mg, about 7 mg to about 30 mg, about 7 mg to about 13 mg, about 8 mg to about 20 mg, about 8 mg to about 12 mg, about 9 mg to about 11 mg, about 20 mg to about 30 mg, about 30 mg to about 40 mg, about 40 mg to about 50 mg, about 10 mg, about 20 mg, about 30 mg, about 40 mg, or about 50 mg of lutein or an equivalent amount of a prodrug, a pharmaceutically acceptable salt, and/or a pharmaceutically acceptable salt of a prodrug of lutein. In some embodiments, the composition comprises about 10 mg of lutein.

In some embodiments, the compositions disclosed herein further comprise zeaxanthin, a prodrug thereof, and/or a pharmaceutically acceptable salt of zeaxanthin. In some embodiments, the zeaxanthin, a prodrug thereof, and/or a pharmaceutically acceptable salt of zeaxanthin is R′,R′-zeaxanthin. Zeaxanthin is a carotenoid. In some embodiments, the composition comprises about 1 mg to about 20 mg, such as about 1.5 mg to about 2.5 mg, of zeaxanthin or an equivalent amount of a prodrug, a pharmaceutically acceptable salt, and/or a pharmaceutically acceptable salt of a prodrug of zeaxanthin. In some embodiments, the composition comprises about 1 mg to about 20 mg, such as about 1 mg to about 3 mg, about 3 mg to about 5 mg, about 1.5 mg to about 2.5 mg, such as about 1.5 mg to about 2 mg, about 2 mg to about 2.5 mg, about 5 mg to about 7 mg, about 7 mg to about 9 mg, about 9 mg to about 11 mg, about 11 mg to about 13 mg, about 13 mg to about 15 mg, about 15 mg to about 17 mg about 17 mg to about 19 mg, about 2 mg, about 3 mg, about 4 mg, about 5, mg, about 10 mg, or about 20 mg of zeaxanthin or an equivalent amount of a prodrug, a pharmaceutically acceptable salt, and/or a pharmaceutically acceptable salt of a prodrug of zeaxanthin. In some embodiments, the composition comprises about 2 mg of zeaxanthin.

In some embodiments, the compositions described herein are dietary or nutritional supplements or pharmaceutical compositions. In some embodiments, the compositions may be in the form of a food product, a component of a food product. In some embodiments, the compositions of the present disclosure may be used in methods for improving the health of a subject. In some embodiments, the compositions described herein are pharmaceutical compositions.

In some embodiments, the composition is formulated in a form suitable for oral, intraocular, intraperitoneal, intravenous, subcutaneous, sublingual, transcutaneous, and/or intramuscular administration. In some embodiments, the intraocular administration is chosen from intravitreal injection and suprachoroidal injection. In some embodiments, the composition is formulated in a form suitable for oral administration. Non-limiting suitable solid oral formulations include tablets, capsules, sachets, lozenges, powders, pills, granules, and pellets. Non-limiting suitable liquid oral formulations include solutions, suspensions, dispersions, emulsions, and oils. In some embodiments, the composition is in liquid, semisolid or solid form. For example, the compositions may be administered as tablets, gel packs, capsules, gelatin capsules, flavored drinks, as a powder that can be reconstituted. In some embodiments, the composition is in the form of a tablet, capsule, soft gel, liquid, or powder. In some embodiments, the composition is a soft gelatin capsule and a hard gelatin capsule.

In some embodiments, the composition is in the form of a chewable oral formulation, such as a chewable tablet.

In some embodiments, the composition is in the form of an immediate release formulation or a modified release formulation, such as a delayed release and/or an extended release formulation.

In some embodiments, the compositions may further comprise at least one pharmaceutically acceptable excipient. Non-limiting examples of suitable excipients include surfactants, humectants, plasticizers, binders, crystallization inhibitors, wetting agents, fillers, solubilizers, bioavailability enhancers, pH adjusting agents, and flavorants. The at least one pharmaceutically acceptable excipient, as used herein, also includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various excipients used in formulating oral compositions and known techniques for the preparation thereof. Except insofar as any conventional carrier is incompatible with the vitamins of this disclosure, such as by producing any undesirable biological effect or otherwise interacting in a deleterious manner with any other component(s) of the composition, its use is contemplated to be within the scope of this disclosure. Non-limiting examples of suitable pharmaceutically acceptable excipients include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium tri silicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

In some embodiments, the compositions may further comprise or be co-administered with at least one additional active ingredient. In some embodiments, the at least one additional therapeutic agent is chosen from anti-inflammatory agents (e.g., anti-IL-6 agent, anti-IL-8 agents, aspirin, ibuprofen, and naproxen), anti-angiogenic agents (e.g., anti-VEGF agents, ranibizumab, bevacizumab, acadesine, and AMPK activators), anti-oxidative agents (e.g., vitamin C, vitamin E, vitamin A, glutathione, catalase, etc.), omega-3 fatty acids (e.g., alpha-linolenic acid (ALA), eicosapentaenoic acid (EPA), and docosahexaenoic acid (DHA)), and vitamins and minerals (e.g., vitamin C, vitamin E, vitamin A, lutein, zeaxanthin, zinc, and copper). In some embodiments, the composition further comprises lutein and zeaxanthin.

In some embodiments, the compositions may be co-administered with at least one additional composition comprising vitamin C, a prodrug thereof, and/or a pharmaceutically acceptable salt of any of the foregoing; vitamin E, a prodrug thereof, and/or a pharmaceutically acceptable salt of any of the foregoing; zinc, a prodrug thereof, and/or a pharmaceutically acceptable form of any of the foregoing; and copper, a prodrug thereof, and/or a pharmaceutically acceptable form of any of the foregoing. In some embodiments, the additional composition comprises 350.0 mg to 800.0 mg of vitamin C or an equivalent amount of a prodrug, a pharmaceutically acceptable salt, and/or a pharmaceutically acceptable salt of a prodrug of vitamin C; 150.0 mg to 550.0 mg of vitamin E or an equivalent amount of a prodrug, a pharmaceutically acceptable salt, and/or a pharmaceutically acceptable salt of a prodrug of vitamin E; 20.0 mg to 90.0 mg of zinc or an equivalent amount of a prodrug, a pharmaceutically acceptable form, and/or a pharmaceutically acceptable salt of a prodrug of zinc; 1.5 mg to 2.5 mg of copper or an equivalent amount of a prodrug, a pharmaceutically acceptable form, and/or a pharmaceutically acceptable salt of a prodrug of copper; 5.0 mg to 50.0 mg of lutein or an equivalent amount of a prodrug, a pharmaceutically acceptable salt, and/or a pharmaceutically acceptable salt of a prodrug of lutein; and/or 1.0 mg to 20.0 mg of zeaxanthin or an equivalent amount of a prodrug, a pharmaceutically acceptable salt, and/or a pharmaceutically acceptable salt of a prodrug of zeaxanthin. In some embodiments, the additional composition comprises about 350 mg to about 800 mg of vitamin C or an equivalent amount of a prodrug, a pharmaceutically acceptable salt, and/or a pharmaceutically acceptable salt of a prodrug of vitamin C; about 50 mg to about 550 mg of vitamin E or an equivalent amount of a prodrug, a pharmaceutically acceptable salt, and/or a pharmaceutically acceptable salt of a prodrug of vitamin E; about 20 mg to about 90 mg of zinc or an equivalent amount of a prodrug, a pharmaceutically acceptable form, and/or a pharmaceutically acceptable salt of a prodrug of zinc; about 1.5 mg to about 2.5 mg of copper or an equivalent amount of a prodrug, a pharmaceutically acceptable form, and/or a pharmaceutically acceptable salt of a prodrug of copper; about 5 mg to about 50 mg of lutein or an equivalent amount of a prodrug, a pharmaceutically acceptable salt, and/or a pharmaceutically acceptable salt of a prodrug of lutein; and/or about 1 mg to about 20 mg of zeaxanthin or an equivalent amount of a prodrug, a pharmaceutically acceptable salt, and/or a pharmaceutically acceptable salt of a prodrug of zeaxanthin.

In some embodiments, the compositions may be co-administered with at least one additional composition comprising vitamin C, a prodrug thereof, and/or a pharmaceutically acceptable salt of any of the foregoing; vitamin E, a prodrug thereof, and/or a pharmaceutically acceptable salt of any of the foregoing; zinc, a prodrug thereof, and/or a pharmaceutically acceptable form of any of the foregoing; and copper, a prodrug thereof, and/or a pharmaceutically acceptable form of any of the foregoing. In some embodiments, the additional composition comprises 350.0 mg to 800.0 mg of vitamin C or an equivalent amount of a prodrug, a pharmaceutically acceptable salt, and/or a pharmaceutically acceptable salt of a prodrug of vitamin C; 150.0 mg to 550.0 mg of vitamin E or an equivalent amount of a prodrug, a pharmaceutically acceptable salt, and/or a pharmaceutically acceptable salt of a prodrug of vitamin E; 20.0 mg to 90.0 mg of zinc or an equivalent amount of a prodrug, a pharmaceutically acceptable form, and/or a pharmaceutically acceptable salt of a prodrug of zinc; 1.5 mg to 2.5 mg of copper or an equivalent amount of a prodrug, a pharmaceutically acceptable form, and/or a pharmaceutically acceptable salt of a prodrug of copper; 5.0 mg to 15.0 mg of lutein or an equivalent amount of a prodrug, a pharmaceutically acceptable salt, and/or a pharmaceutically acceptable salt of a prodrug of lutein; and/or 1.5 mg to 2.5 mg of zeaxanthin or an equivalent amount of a prodrug, a pharmaceutically acceptable salt, and/or a pharmaceutically acceptable salt of a prodrug of zeaxanthin. In some embodiments, the additional composition comprises about 350 mg to about 800 mg of vitamin C or an equivalent amount of a prodrug, a pharmaceutically acceptable salt, and/or a pharmaceutically acceptable salt of a prodrug of vitamin C; about 50 mg to about 550 mg of vitamin E or an equivalent amount of a prodrug, a pharmaceutically acceptable salt, and/or a pharmaceutically acceptable salt of a prodrug of vitamin E; about 20 mg to about 90 mg of zinc or an equivalent amount of a prodrug, a pharmaceutically acceptable form, and/or a pharmaceutically acceptable salt of a prodrug of zinc; about 1.5 mg to about 2.5 mg of copper or an equivalent amount of a prodrug, a pharmaceutically acceptable form, and/or a pharmaceutically acceptable salt of a prodrug of copper; about 5 mg to about 15 mg of lutein or an equivalent amount of a prodrug, a pharmaceutically acceptable salt, and/or a pharmaceutically acceptable salt of a prodrug of lutein; and/or about 1.5 mg to about 2.5 mg of zeaxanthin or an equivalent amount of a prodrug, a pharmaceutically acceptable salt, and/or a pharmaceutically acceptable salt of a prodrug of zeaxanthin.

Combination of the disclosed compositions and at least one additional active ingredient may advantageously produce one or more of the following effects: (1) additive and/or synergistic benefits; (2) reduction of the side effects and/or adverse effects associated with use of the prescription medicine in the absence of the compositions disclosed herein; and/or (3) the ability to lower the dosage of the prescription medicine in comparison to the amount of prescription medicine needed in the absence of the compositions disclosed herein.

As disclosed above, in some embodiments, the compositions may further comprise or be co-administered with at least one additional active ingredient. In some embodiments, the at least one additional active ingredient may be chosen from vitamins and minerals. In some embodiments, the at least one additional active ingredient is chosen from

vitamin C, prodrugs thereof, and pharmaceutically acceptable salts of any of the foregoing;

vitamin E prodrugs thereof, and pharmaceutically acceptable salts of any of the foregoing;

zinc, prodrugs thereof, and pharmaceutically acceptable salts of any of the foregoing;

copper, prodrugs thereof, and pharmaceutically acceptable salts of any of the foregoing;

vitamin B1, prodrugs thereof, and pharmaceutically acceptable salts of any of the foregoing;

vitamin B2, prodrugs thereof, and pharmaceutically acceptable salts of any of the foregoing;

vitamin B3, prodrugs thereof, and pharmaceutically acceptable salts of any of the foregoing;

vitamin B5, prodrugs thereof, and pharmaceutically acceptable salts of any of the foregoing;

vitamin B6, prodrugs thereof, and pharmaceutically acceptable salts of any of the foregoing;

vitamin B7, prodrugs thereof, and pharmaceutically acceptable salts of any of the foregoing;

vitamin B9, prodrugs thereof, and pharmaceutically acceptable salts of any of the foregoing;

vitamin B12, prodrugs thereof, and pharmaceutically acceptable salts of any of the foregoing;

lutein, prodrugs thereof, and pharmaceutically acceptable salts of any of the foregoing; and

zeaxanthin, prodrugs thereof, and pharmaceutically acceptable salts of any of the foregoing.

In some embodiments, the vitamin C, prodrugs thereof, and pharmaceutically acceptable salts of any of the foregoing comprises ascorbic acid. In some embodiments, the vitamin E, prodrugs thereof, and pharmaceutically acceptable salts of any of the foregoing comprises dl-alpha tocopheryl acetate and/or alpha-tocopherol. In some embodiments, the zinc, prodrugs thereof, and pharmaceutically acceptable salts of any of the foregoing comprises zinc oxide and/or zinc gluconate. In some embodiments, the copper, prodrugs thereof, and pharmaceutically acceptable salts of any of the foregoing comprises copper oxide and/or copper gluconate. In some embodiments, the vitamin B1, prodrugs thereof, and pharmaceutically acceptable salts of any of the foregoing comprises thiamin. In some embodiments, the vitamin B2, prodrugs thereof, and pharmaceutically acceptable salts of any of the foregoing comprises riboflavin. In some embodiments, the vitamin B3, prodrugs thereof, and pharmaceutically acceptable salts of any of the foregoing comprises nicotinic acid and/or niacinamide. In some embodiments, the vitamin B5, prodrugs thereof, and pharmaceutically acceptable salts of any of the foregoing comprises pantothenic acid. In some embodiments, the vitamin B6, prodrugs thereof, and pharmaceutically acceptable salts of any of the foregoing comprises pyridoxine. In some embodiments, the lutein, prodrugs thereof, and pharmaceutically acceptable salts of any of the foregoing comprises lutein (R′R′). In some embodiments, the zeaxanthin, prodrugs thereof, and pharmaceutically acceptable salts of any of the foregoing comprises zeaxanthin (R′R′). In some embodiments, the vitamin B7, prodrugs thereof, and pharmaceutically acceptable salts of any of the foregoing comprises biotin. In some embodiments, the vitamin B9, prodrugs thereof, and pharmaceutically acceptable salts of any of the foregoing comprises folic acid and/or methyl tetrahydrofolate. In some embodiments, the vitamin B12, prodrugs thereof, and pharmaceutically acceptable salts of any of the foregoing comprise cyanocobalamin.

In some embodiments, the composition further comprises vitamin C, optionally in the form of ascorbic acid; vitamin E optionally in the form of dl-alpha tocopheryl acetate; zinc, optionally in the form of zinc oxide; thiamin; copper, optionally in the form of copper oxide; riboflavin; vitamin B3, optionally in the form of nicotinic acid and/or niacinamide; pyridoxine; biotin, folate, folic acid and/or methyl tetrahydrofolate; and vitamin B12, optionally in the form of cyanocobalamin. In some embodiments, the composition further comprises R′R′ lutein and R′R zeaxanthin.

In some embodiments, the vitamin C, prodrugs thereof, and pharmaceutically acceptable salts of any of the foregoing comprises ascorbic acid. In some embodiments, the vitamin C, prodrugs thereof, and pharmaceutically acceptable salts of any of the foregoing is ascorbic acid. The U.S. recommended dietary allowance (RDA) for vitamin C in the form of ascorbic acid is 60 mg. In some embodiments, the composition further comprises 60 mg to 850 mg of vitamin C or an equivalent amount of a prodrug, a pharmaceutically acceptable salt, and/or a pharmaceutically acceptable salt of a prodrug of vitamin C. In some embodiments, the composition further comprises 100.0 mg to 800.0 mg, such as 150.0 mg to 800.0 mg, 250.0 mg to 800.0 mg, 250.0 mg to 750.0 mg, 500.0 mg to 775.0 mg, 600.0 mg to 750.0 mg, 500.0 mg, or 750.0 mg, of vitamin C or an equivalent amount of a prodrug, a pharmaceutically acceptable salt, and/or a pharmaceutically acceptable salt of a prodrug of vitamin C. In some embodiments, the composition comprises 500.0 mg of vitamin C, optionally in the form of ascorbic acid. In some embodiments, the composition further comprises about 60 mg to about 850 mg of vitamin C or an equivalent amount of a prodrug, a pharmaceutically acceptable salt, and/or a pharmaceutically acceptable salt of a prodrug of vitamin C. In some embodiments, the composition further comprises about 100 mg to about 800 mg, such as about 150 mg to about 800 mg, about 250 mg to about 800 mg, about 250 mg to about 750 mg, about 500 mg to about 775 mg, about 600 mg to about 750 mg, about 500 mg, or about 750 mg, of vitamin C or an equivalent amount of a prodrug, a pharmaceutically acceptable salt, and/or a pharmaceutically acceptable salt of a prodrug of vitamin C. In some embodiments, the composition comprises about 500 mg of vitamin C, optionally in the form of ascorbic acid.

In some embodiments, the vitamin E, prodrugs thereof, and pharmaceutically acceptable salts of any of the foregoing comprises alpha-tocopherol. In some embodiments, the vitamin E, prodrugs thereof, and pharmaceutically acceptable salts of any of the foregoing is dl-alpha tocopheryl acetate. The RDA of vitamin E is 15.0 mg. No adverse effects of vitamin E have been observed at levels as high as 800.0 mg. In some embodiments, the composition further comprises 15.0 mg to 800 mg vitamin E or an equivalent amount of a prodrug, a pharmaceutically acceptable salt, and/or a pharmaceutically acceptable salt of a prodrug of vitamin E. In some embodiments, the composition further comprises 15.0 mg to 800.0 mg vitamin E, such as 50.0 mg to 750.0 mg, 90.0 mg to 600.0 mg, 180.0 mg to 500.0 mg, 250.0 mg to 400.0 mg, 300.0 mg to 350.0 mg vitamin E or an equivalent amount of a prodrug, a pharmaceutically acceptable salt, and/or a pharmaceutically acceptable salt of a prodrug of vitamin E. In some embodiments, the composition further comprises 180 mg vitamin E. In some embodiments, the composition further comprises about 15 mg to about 800 mg vitamin E or an equivalent amount of a prodrug, a pharmaceutically acceptable salt, and/or a pharmaceutically acceptable salt of a prodrug of vitamin E. In some embodiments, the composition further comprises about 15 mg to about 800 mg vitamin E, such as about 50 mg to about 750 mg, about 90 mg to about 600 mg, about 180 mg to about 500 mg, about 250 mg to about 400 mg, about 300 mg to about 350 mg vitamin E or an equivalent amount of a prodrug, a pharmaceutically acceptable salt, and/or a pharmaceutically acceptable salt of a prodrug of vitamin E. In some embodiments, the composition further comprises about 180 mg vitamin E.

In some embodiments, the zinc, a prodrug thereof, and/or a pharmaceutically acceptable form of any of the foregoing comprises zinc oxide. In some embodiments, the zinc, a prodrug thereof, and/or a pharmaceutically acceptable form of any of the foregoing is zinc oxide. The RDA for zinc is about 15 mg. In some embodiments, the composition further comprises 15 mg to 100 mg of zinc or an equivalent amount of a prodrug, a pharmaceutically acceptable salt, and/or a pharmaceutically acceptable form of a prodrug of zinc. In some embodiments, the composition further comprises 15.0 mg to 100.0 mg, such as 40.0 mg to 95.0 mg, 50.0 mg to 90.0 mg, 60.0 mg to 85.0 mg, 70.0 mg to 80.0 mg, or 80.0 mg, of zinc or an equivalent amount of a prodrug, a pharmaceutically acceptable form, and/or a pharmaceutically acceptable salt of a prodrug of zinc. In some embodiments, the composition further comprises 80.0 mg of zinc, optionally in the form of zinc oxide. In some embodiments, the composition further comprises 25.0 mg zinc, optionally in the form of oxide. In some embodiments, the composition further comprises about 15 mg to about 100 mg of zinc, optionally in the form of zinc oxide, or an equivalent amount of a prodrug, a pharmaceutically acceptable salt, and/or a pharmaceutically acceptable form of a prodrug of zinc. In some embodiments, the composition further comprises about 15 mg to about 100 mg, such as about 40 mg to about 95 mg, about 50 mg to about 90 mg, about 60 mg to about 85.0 mg, about 70 mg to about 80 mg, or about 80 mg, zinc or an equivalent amount of a prodrug, a pharmaceutically acceptable form, and/or a pharmaceutically acceptable salt of a prodrug of zinc. In some embodiments, the composition further comprises about 80 mg of zinc, optionally in the form of zinc oxide. In some embodiments, the composition further comprises about 25 mg zinc, optionally in the form of zinc oxide.

In some embodiments, the copper, a prodrug thereof, and/or a pharmaceutically acceptable form of any of the foregoing comprises copper oxide. In some embodiments, the copper, a prodrug thereof, and/or a pharmaceutically acceptable form of any of the foregoing is copper oxide. The RDA for copper is 2.0 mg. In some embodiments, the composition further comprises 1.5 mg to 2.5 mg of copper or an equivalent amount of a prodrug, a pharmaceutically acceptable form, and/or a pharmaceutically acceptable salt of a prodrug of copper. In some embodiments, the composition further comprises 1.5 mg to 2.0 mg, such as 2.0 mg, of copper or an equivalent amount of a prodrug, a pharmaceutically acceptable form, and/or a pharmaceutically acceptable salt of a prodrug of copper. In some embodiments, the composition further comprises 2.0 mg of copper, optionally in the form of copper oxide. In some embodiments, the composition further comprises about 1.5 mg to about 2.5 mg of copper or an equivalent amount of a prodrug, a pharmaceutically acceptable form, and/or a pharmaceutically acceptable salt of a prodrug of copper. In some embodiments, the composition further comprises about 1.5 mg to about 2.0 mg, such as about 2.0 mg, of copper or an equivalent amount of a prodrug, a pharmaceutically acceptable form, and/or a pharmaceutically acceptable salt of a prodrug of copper. In some embodiments, the composition further comprises about 2.0 mg of copper, optionally in the form of copper oxide.

In some embodiments, the vitamin B1, prodrugs thereof, and pharmaceutically acceptable salts of any of the foregoing comprises thiamin. In some embodiments, the vitamin B1, prodrugs thereof, and pharmaceutically acceptable salts of any of the foregoing is thiamin. Vitamin B1 is a water soluble vitamin. The U.S. recommended dietary allowance (RDA) for vitamin B1 taken by mouth is 1.2 mg for males and 1.1 mg for females over the age of 18. In some embodiments, the composition further comprises 0.1 mg to 25 mg of vitamin B1 or an equivalent amount of a prodrug, a pharmaceutically acceptable salt, and/or a pharmaceutically acceptable salt of a prodrug of vitamin B1. In some embodiments, the composition further comprises 0.1 mg to 25.0 mg of thiamin or an equivalent amount of a prodrug, a pharmaceutically acceptable salt, and/or a pharmaceutically acceptable salt of a prodrug of vitamin B1. In some embodiments, the composition further comprises 0.1 mg to 25.0 mg, such as 0.5 mg to 20.0 mg, 1.0 mg to 15.0 mg, 1.5 mg to 10.0 mg, 1.1 mg to 4.5 mg, 1.2 mg to 3.5 mg, 1.2 mg to 2.5 mg, 1.2 mg to 1.8 mg, 1.5 mg, 2.0 to 20.0 mg, 2.0 mg to 15.0 mg, 2.5 mg to 10.0 mg, 2.5 mg to 3.5 mg, 2.6 mg to 3.4 mg, 2.8 mg to 3.2 mg, 3 mg, 3.0 mg to 20.0 mg, 3.0 mg to 15.0 mg, 3.0 mg to 10.0 mg, 3.0 mg to 5.0 mg, 3.2 mg to 4.8 mg, 3.4 mg to 4.6 mg, or 4.5 mg, of thiamin or an equivalent amount of a prodrug, a pharmaceutically acceptable salt, and/or a pharmaceutically acceptable salt of a prodrug of vitamin B1. In some embodiments, the composition further comprises 1.5 mg of thiamin. In some embodiments, the composition further comprises 3.0 mg of thiamin. In some embodiments, the composition further comprises 4.5 mg thiamin. In some embodiments, the composition further comprises about 0.1 mg to about 25 mg of vitamin B1 or an equivalent amount of a prodrug, a pharmaceutically acceptable salt, and/or a pharmaceutically acceptable salt of a prodrug of vitamin B1. In some embodiments, the composition further comprises about 0.1 mg to about 25 mg of thiamin or an equivalent amount of a prodrug, a pharmaceutically acceptable salt, and/or a pharmaceutically acceptable salt of a prodrug of vitamin B1. In some embodiments, the composition further comprises about 0.1 mg to about 25 mg, such as about 0.5 mg to about 20 mg, about 1 mg to about 15 mg, about 1.5 mg to about 10 mg, about 1.1 mg to about 4.5 mg, about 1.2 mg to about 3.5 mg, about 1.2 mg to about 2.5 mg, about 1.2 mg to about 1.8 mg, about 1.5 mg, about 2 to about 20 mg, about 2 mg to about 15 mg, about 2.5 mg to about 10 mg, about 2.5 mg to about 3.5 mg, about 2.6 mg to about 3.4 mg, about 2.8 mg to about 3.2 mg, about 3 mg, about 3 mg to about 20 mg, about 3 mg to about 15 mg, about 3 mg to about 10 mg, about 3 mg to about 5 mg, about 3.2 mg to about 4.8 mg, about 3.4 mg to about 4.6 mg or about 4.5 mg, of thiamin or an equivalent amount of a prodrug, a pharmaceutically acceptable salt, and/or a pharmaceutically acceptable salt of a prodrug of vitamin B1. In some embodiments, the composition further comprises about 1.5 mg of thiamin. In some embodiments, the composition further comprises about 3 mg of thiamin. In some embodiments, the composition further comprises about 4.5 mg thiamin.

In some embodiments, the vitamin B2, prodrugs thereof, and pharmaceutically acceptable salts of any of the foregoing comprises riboflavin. In some embodiments, the vitamin B2, prodrugs thereof, and pharmaceutically acceptable salts of any of the foregoing is riboflavin. Vitamin B2 is a water soluble vitamin. The U.S. recommended dietary allowance (RDA) for vitamin B2 taken by mouth is 1.1 mg for males and 1.3 mg for females over the age of 18. In some embodiments, the composition further comprises 0.1 mg to 25.0 mg of riboflavin or an equivalent amount of a prodrug, a pharmaceutically acceptable salt, and/or a pharmaceutically acceptable salt of a prodrug of vitamin B2. In some embodiments, the composition further comprises 0.1 mg to 25.0 mg, such as 1.0 mg to 20.0 mg, 1.0 mg to 15.0 mg, 1.0 mg to 10.0 mg, 1.1 mg to 4.5 mg, 1.2 mg to 3.5 mg, 1.2 mg to 2.5 mg, 1.3 mg to 1.8 mg, 1.7 mg, 2.0 mg to 20.0 mg, 2.0 mg to 15.0 mg, 2.0 mg to 10.0 mg, 2.0 mg to 4.0 mg, 2.2 mg to 3.8 mg, 2.6 mg to 3.4 mg, 3.4 mg, 3.0 mg to 20.0 mg, 3.0 mg to 15.0 mg, 3.0 mg to 10.0 mg, 3.0 mg to 6.0 mg, 3.2 mg to 5.8 mg, 3.4 mg to 5.6 mg or 5.1 mg of riboflavin or an equivalent amount of a prodrug, a pharmaceutically acceptable salt, and/or a pharmaceutically acceptable salt of a prodrug of vitamin B2. In some embodiments, the composition further comprises 1.7 mg of riboflavin. In some embodiments, the composition further comprises 3.4 mg of riboflavin. In some embodiments, the composition further comprises 5.1 mg riboflavin. In some embodiments, the composition further comprises about 0.1 mg to about 25 mg of riboflavin or an equivalent amount of a prodrug, a pharmaceutically acceptable salt, and/or a pharmaceutically acceptable salt of a prodrug of vitamin B2. In some embodiments, the composition further comprises about 0.1 mg to about 25 mg, such as about 1.0 mg to about 20 mg, about 1 mg to about 15 mg, about 1 mg to about 10 mg, about 1.1 mg to about 4.5 mg, about 1.2 mg to about 3.5 mg, about 1.2 mg to about 2.5 mg, about 1.3 mg to about 1.8 mg, about 1.7 mg, about 2 mg to about 20 mg, about 2 mg to about 15 mg, about 2 mg to about 10 mg, about 2 mg to about 4 mg, about 2.2 mg to about 3.8 mg, about 2.6 mg to about 3.4 mg, about 3.4 mg, about 3 mg to about 20 mg, about 3 mg to about 15 mg, about 3 mg to about 10 mg, about 3 mg to about 6 mg, about 3.2 mg to about 5.8 mg, about 3.4 mg to about 5.6 mg, or about 5.1 mg, of riboflavin or an equivalent amount of a prodrug, a pharmaceutically acceptable salt, and/or a pharmaceutically acceptable salt of a prodrug of vitamin B2. In some embodiments, the composition further comprises about 1.7 mg of riboflavin. In some embodiments, the composition further comprises about 3.4 mg of riboflavin. In some embodiments, the composition further comprises about 5.1 mg of riboflavin.

In some embodiments, the vitamin B3, prodrugs thereof, and pharmaceutically acceptable salts of any of the foregoing comprises nicotinic acid, and/or niacinamide. In some embodiments, the vitamin B3, prodrugs thereof, and pharmaceutically acceptable salts of any of the foregoing is nicotinic acid. Vitamin B3 is a water soluble vitamin of which there are three chemical forms: nicotinic acid, nicotinamide, and niacinamide. The U.S. recommended dietary allowance (RDA) for vitamin B3 taken by mouth is 16 mg for males and 14 mg for females over the age of 14. In some embodiments, the composition further comprises 1.0 mg to 300.0 mg, such as 5.0 mg to 150.0 mg, 10.0 mg to 100.0 mg, 20.0 mg to 60.0 mg, of vitamin B3 or an equivalent amount of a prodrug, a pharmaceutically acceptable salt, and/or a pharmaceutically acceptable salt of a prodrug of vitamin B3. In some embodiments, the composition further comprises 18.0 mg to 25.0 mg, such as 18.5 mg to 24.5 mg, 17.0 mg to 24.0 mg, 16.5 mg to 23.5 mg, 16.0 mg to 23.0 mg, or 16.0 mg to 22.0 mg, of vitamin B3 or an equivalent amount of a prodrug, a pharmaceutically acceptable salt, and/or a pharmaceutically acceptable salt of a prodrug of vitamin B3. In some embodiments, the composition further comprises 20.0 mg of vitamin B3. In some embodiments, the composition further comprises 40.0 mg of vitamin B3. In some embodiments, the composition further comprises 60.0 mg of vitamin B3. In some embodiments, the composition further comprises about 1.0 mg to about 300.0 mg, such as about 5.0 mg to about 150.0 mg, about 10.0 mg to about 100.0 mg, about 20.0 mg to about 60.0 mg, of vitamin B3 or an equivalent amount of a prodrug, a pharmaceutically acceptable salt, and/or a pharmaceutically acceptable salt of a prodrug of vitamin B3. In some embodiments, the composition further comprises about 18 mg to about 25 mg, such as about 18.5 mg to about 24.5 mg, about 17 mg to about 24 mg, about 16.5 mg to about 23.5 mg, about 16 mg to about 23 mg, or about 16 mg to about 22 mg, of vitamin B3 or an equivalent amount of a prodrug, a pharmaceutically acceptable salt, and/or a pharmaceutically acceptable salt of a prodrug of vitamin B3. In some embodiments, the composition further comprises about 20 mg of vitamin B3. In some embodiments, the composition further comprises about 40 mg of vitamin B3. In some embodiments, the composition further comprises about 60 g of vitamin B3.

In some embodiments, the vitamin B5, prodrugs thereof, and pharmaceutically acceptable salts of any of the foregoing comprises pantothenic acid. In some embodiments, the vitamin B5, prodrugs thereof, and pharmaceutically acceptable salts of any of the foregoing is pantothenic acid. The recommended dietary allowance (RDA) for vitamin B5 is 5 mg for people over the age of 14. In some embodiments, the composition further comprises 1.0 mg to 250.0 mg of pantothenic acid or an equivalent amount of a prodrug, a pharmaceutically acceptable salt, and/or a pharmaceutically acceptable salt of a prodrug of vitamin B5. In some embodiments, the composition further comprises 1.0 mg to 250.0 mg, such as 2.0 mg to 200.0 mg, 5.0 mg to 150.0 mg, 5.0 mg to 100.0 mg, 5.0 mg to 50.0 mg, 5.0 mg to 30.0 mg, 4.5 mg to 13.5 mg, 5.0 mg to 12.5 mg, 5.5 mg to 12.0 mg, 6.0 mg to 12.5 mg, 6.5 mg to 12.0 mg, 8.5 mg to 11.0 mg, 9.0 to 10.5 mg, or 10.0 mg, 10.0 mg to 25.0 mg, 10.5 mg to 24.5 mg, 12.0 mg to 22.0 mg, 20.0 mg, 20.0 mg to 45.0 mg, 25.0 mg to 35.0 mg, 28.0 mg to 32.0 mg or 30.0 mg, of vitamin B5 or an equivalent amount of a prodrug, a pharmaceutically acceptable salt, and/or a pharmaceutically acceptable salt of a prodrug of vitamin B5. In some embodiments, the composition further comprises 10.0 mg of pantothenic acid. In some embodiments, the composition further comprises 20.0 mg of panthothenic acid. In some embodiments, the composition further comprises 30.0 mg of panthothenic acid. In some embodiments, the composition further comprises about 1 mg to about 250 mg of pantothenic acid or an equivalent amount of a prodrug, a pharmaceutically acceptable salt, and/or a pharmaceutically acceptable salt of a prodrug of vitamin B5. In some embodiments, the composition further comprises about 1 mg to about 250 mg, such as about 2 mg to about 200 mg, about 5 mg to about 150 mg, about 5 mg to about 100 mg, about 5 mg to about 50 mg, about 5 mg to about 30 mg, about 4.5 mg to about 13.5 mg, about 5.0 mg to about 12.5 mg, about 5.5 mg to about 12 mg, about 6 mg to about 12.5 mg, about 6.5 mg to about 12 mg, about 8.5 mg to about 11.0 mg, about 9.0 to about 10.5 mg, or about 10 mg, about 10 mg to about 25 mg, about 10.5 mg to about 24.5 mg, about 12 mg to about 22 mg, about 20 mg, about 20 mg to about 45 mg, about 25 mg to about 35 mg, about 28 mg to about 32 mg or about 30 mg, of vitamin B5 or an equivalent amount of a prodrug, a pharmaceutically acceptable salt, and/or a pharmaceutically acceptable salt of a prodrug of vitamin B5. In some embodiments, the composition further comprises about 10 mg of pantothenic acid. In some embodiments, the composition further comprises about 20 mg of panthothenic acid. In some embodiments, the composition further comprises about 30 mg of panthothenic acid.

In some embodiments, the vitamin B6, prodrugs thereof, and pharmaceutically acceptable salts of any of the foregoing comprises pyridoxine. In some embodiments, the vitamin B6, prodrugs thereof, and pharmaceutically acceptable salts of any of the foregoing is pyridoxine. The recommended dietary allowance (RDA) for vitamin B6 is 1.3 mg for people aged 14 to 50. In some embodiments, the composition further comprises 25.0 mg to 100.0 mg of pyridoxine or an equivalent amount of a prodrug, a pharmaceutically acceptable salt, and/or a pharmaceutically acceptable salt of a prodrug of vitamin B6. In some embodiments, the composition further comprises 25.0 mg to 200.0 mg of vitamin B6, such as 25.0 mg to 100.0 mg, 30.0 mg to 90.0 mg, 40.0 mg to 80.0 mg, 45.0 mg to 70.0 mg, 45.0 mg to 60.0 mg, 55.0 mg, or 50.0 mg, of vitamin B6 or an equivalent amount of a prodrug, a pharmaceutically acceptable salt, and/or a pharmaceutically acceptable salt of a prodrug of vitamin B6. In some embodiments, the composition further comprises 50.0 mg of pyridoxine. In some embodiments, the composition further comprises about 25 mg to about 100 mg of pyridoxine or an equivalent amount of a prodrug, a pharmaceutically acceptable salt, and/or a pharmaceutically acceptable salt of a prodrug of vitamin B6. In some embodiments, the composition further comprises about 25 mg to about 200 mg of vitamin B6, such as about 25 mg to about 100 mg, about 30 mg to about 90 mg, about 40 mg to about 80 mg, about 45 mg to about 70 mg, about 45 mg to about 60 mg, about 55 mg, or about 50 mg, of vitamin B6 or an equivalent amount of a prodrug, a pharmaceutically acceptable salt, and/or a pharmaceutically acceptable salt of a prodrug of vitamin B6. In some embodiments, the composition further comprises about 50 mg of pyridoxine.

In some embodiments, the vitamin B7, prodrugs thereof, and pharmaceutically acceptable salts of any of the foregoing comprises biotin. In some embodiments, the vitamin B7, prodrugs thereof, and pharmaceutically acceptable salts of any of the foregoing is biotin. The recommended dietary allowance (RDA) for vitamin B7 is 0.03 mg for people over the age of 18. In some embodiments, the composition further comprises 0.01 mg to 1.0 mg of biotin or an equivalent amount of a prodrug, a pharmaceutically acceptable salt, and/or a pharmaceutically acceptable salt of a prodrug of vitamin B7. In some embodiments, the composition further comprises 0.01 mg to 1.0 mg, such as 0.01 mg to 0.75 mg, 0.02 mg to 0.75 mg, 0.02 mg to 0.05 mg, 0.02 mg to 0.04 mg, or 0.03 mg, of vitamin B7 or an equivalent amount of a prodrug, a pharmaceutically acceptable salt, and/or a pharmaceutically acceptable salt of a prodrug of vitamin B7. In some embodiments, the composition further comprises 0.03 mg of biotin. In some embodiments, the composition further comprises 0.06 mg of biotin. In some embodiments, the composition further comprises 0.09 mg of biotin. In some embodiments, the composition further comprises about 0.01 mg to about 1 mg of biotin or an equivalent amount of a prodrug, a pharmaceutically acceptable salt, and/or a pharmaceutically acceptable salt of a prodrug of vitamin B7. In some embodiments, the composition further comprises about 0.01 mg to about 1 mg, such as about 0.01 mg to about 0.75 mg, about 0.02 mg to about 0.75 mg, about 0.02 mg to about 0.05 mg, about 0.02 mg to about 0.04 mg, or about 0.03 mg, of vitamin B7 or an equivalent amount of a prodrug, a pharmaceutically acceptable salt, and/or a pharmaceutically acceptable salt of a prodrug of vitamin B7. In some embodiments, the composition further comprises about 0.03 mg of biotin. In some embodiments, the composition further comprises about 0.06 mg of biotin. In some embodiments, the composition further comprises about 0.09 mg of biotin.

In some embodiments, the vitamin B9, a prodrug thereof, and/or a pharmaceutically acceptable salt of any vitamin B9 comprises folic acid. In some embodiments, the vitamin B9, a prodrug thereof, and/or a pharmaceutically acceptable salt of any vitamin B9 comprises folate. In some embodiments, the vitamin B9, a prodrug thereof, and/or a pharmaceutically acceptable salt of any vitamin B9 comprises methyl tetrahydrofolate. In some embodiments, the vitamin B9, a prodrug thereof, and/or a pharmaceutically acceptable salt of any vitamin B9 comprises folic acid, folate, and/or methyl tetrahydrofolate. The recommended dietary allowance (RDA) for vitamin B9 is 0.4 mg for people over the age of 19. In some embodiments, the composition further comprises 0.1 mg to 50.0 mg of folic acid, folate and/or methyl tetrahydrofolate or an equivalent amount of a prodrug, a pharmaceutically acceptable salt, and/or a pharmaceutically acceptable salt of a prodrug of vitamin B9. In some embodiments, the composition further comprises 1.0 mg to 3.0 mg of folate, folic acid, and/or methyl tetrahydrofolate or an equivalent amount of a prodrug, a pharmaceutically acceptable salt, and/or a pharmaceutically acceptable salt of a prodrug of vitamin B9. In some embodiments, the composition further comprises 1.0 mg to 3.0 mg, such as 1.1 mg to 2.9 mg, 1.2 mg to 2.8 mg, 1.3 mg to 2.7 mg, 1.4 mg to 2.6 mg, 1.5 mg to 2.6 mg, 1.6 mg to 2.6 mg, 1.7 mg to 2.6 mg, 1.8 mg to 2.6 mg, 1.9 mg to 2.6 mg, 2.0 mg to 2.6 mg, 2.1 mg to 2.6 mg, or 2.5 mg of vitamin B9 or an equivalent amount of a prodrug, a pharmaceutically acceptable salt, and/or a pharmaceutically acceptable salt of a prodrug of vitamin B9. In some embodiments, the composition further comprises 2.5 mg of folic acid and/or methyl tetrahydrofolate. In some embodiments, the composition further comprises about 0.1 mg to about 50 mg of folic acid and/or methyl tetrahydrofolateor an equivalent amount of a prodrug, a pharmaceutically acceptable salt, and/or a pharmaceutically acceptable salt of a prodrug of vitamin B9. In some embodiments, the composition further comprises about 1 mg to about 3 mg of folate, folic acid, and/or methyl tetrahydrofolate or an equivalent amount of a prodrug, a pharmaceutically acceptable salt, and/or a pharmaceutically acceptable salt of a prodrug of vitamin B9. In some embodiments, the composition further comprises about 1 mg to about 3 mg, such as about 1.1 mg to about 2.9 mg, about 1.2 mg to about 2.8 mg, about 1.3 mg to about 2.7 mg, about 1.4 mg to about 2.6 mg, about 1.5 mg to about 2.6 mg, about 1.6 mg to about 2.6 mg, about 1.7 mg to about 2.6 mg, about 1.8 mg to about 2.6 mg, about 1.9 mg to about 2.6 mg, about 2 mg to about 2.6 mg, about 2.1 mg to about 2.6 mg, or about 2.5 mg of vitamin B9 or an equivalent amount of a prodrug, a pharmaceutically acceptable salt, and/or a pharmaceutically acceptable salt of a prodrug of vitamin B9. In some embodiments, the composition further comprises about 2.5 mg of folic acid and/or methyl tetrahydrofolate.

The composition further comprises vitamin B12. The recommended dietary allowance (RDA) for vitamin B12 is 2.4 micrograms (0.0024 mg) for people over the age of 14. In some embodiments, the vitamin B12, a prodrug thereof, and/or a pharmaceutically acceptable salt of vitamin B12 is cyanocobalamin. In some embodiments, the composition further comprises 0.25 mg to 3 mg of vitamin B12 or an equivalent amount of a prodrug, a pharmaceutically acceptable salt, and/or a pharmaceutically acceptable salt of a prodrug of vitamin B12. In some embodiments, the composition further comprises 0.5 mg to 1.5 mg of vitamin B12 or an equivalent amount of a prodrug, a pharmaceutically acceptable salt, and/or a pharmaceutically acceptable salt of a prodrug of vitamin B12. In some embodiments, the composition further comprises 1 mg of vitamin B12. In some embodiments, the composition further comprises about 0.25 mg to about 3 mg of vitamin B12 or an equivalent amount of a prodrug, a pharmaceutically acceptable salt, and/or a pharmaceutically acceptable salt of a prodrug of vitamin B12. In some embodiments, the composition further comprises about 0.5 mg to about 1.5 mg of vitamin B12 or an equivalent amount of a prodrug, a pharmaceutically acceptable salt, and/or a pharmaceutically acceptable salt of a prodrug of vitamin B12. In some embodiments, the composition further comprises about 1 mg of vitamin B12

In some embodiments, the composition further comprises lutein, a prodrug thereof, and/or a pharmaceutically acceptable salt of lutein. In some embodiments, the lutein, a prodrug thereof, and/or a pharmaceutically acceptable salt of lutein is R′,R′-lutein. lutein is a carotenoid. In some embodiments, the composition further comprises 1 mg to 50 mg, such as 5.0 mg to 15.0 mg, of lutein or an equivalent amount of a prodrug, a pharmaceutically acceptable salt, and/or a pharmaceutically acceptable salt of a prodrug of lutein. In some embodiments, the composition further comprises at least 3.0 mg, such as 3.0 mg to 50.0 mg, 5.0 mg to 50.0 mg, 6.0 mg to 40.0 mg, 7.0 mg to 30.0 mg, 8.0 mg to 20.0 mg, 9.0 mg to 11.0 mg, 20.0 mg to 30.0 mg, 30.0 mg to 40.0 mg, 40.0 mg to 50.0 mg, 10.0 mg, 20.0 mg, 30.0 mg, 40.0 mg, or 50.0 mg, of lutein or an equivalent amount of a prodrug, a pharmaceutically acceptable salt, and/or a pharmaceutically acceptable salt of a prodrug of lutein. In some embodiments, the composition further comprises 10.0 mg of lutein. In some embodiments, the composition further comprises about 5 mg to about 50 mg of lutein or an equivalent amount of a prodrug, a pharmaceutically acceptable salt, and/or a pharmaceutically acceptable salt of a prodrug of lutein. In some embodiments, the composition further comprises about 1 mg to about 50 mg, such as about 3 mg to about 50 mg, about 5 mg to about 50 mg, about 6 mg to about 40 mg, about 7 mg to about 30 mg, about 8 mg to about 20 mg, about 5 mg to about 15 mg, such as about 6 mg to about 14 mg, about 7 mg to about 13 mg, about 8 mg to about 12 mg, about 9.0 mg to about 11 mg, or about 10 mg, about 9 mg to about 11 mg, about 20 mg to about 30 mg, about 30 mg to about 40 mg, about 40 mg to about 50 mg, about 10 mg, about 20 mg, about 30 mg, about 40 mg, or about 50 mg, of lutein or an equivalent amount of a prodrug, a pharmaceutically acceptable salt, and/or a pharmaceutically acceptable salt of a prodrug of lutein. In some embodiments, the composition further comprises about 10 mg of lutein.

In some embodiments, the composition further comprises zeaxanthin, a prodrug thereof, and/or a pharmaceutically acceptable salt of zeaxanthin. In some embodiments, the zeaxanthin, a prodrug thereof, and/or a pharmaceutically acceptable salt of zeaxanthin is R′,R′-zeaxanthin. Zeaxanthin is a carotenoid. In some embodiments, the composition further comprises 1.0 mg to 20.0 mg 1.5 mg to 2.5 mg of zeaxanthin or an equivalent amount of a prodrug, a pharmaceutically acceptable salt, and/or a pharmaceutically acceptable salt of a prodrug of zeaxanthin. In some embodiments, the composition further comprises 1.0 mg to 20.0 mg, such as 1.0 mg to 3.0 mg, 3.0 mg to 5.0 mg, 5.0 mg to 7.0 mg, 7.0 mg to 9.0 mg, 9.0 mg to 11.0 mg, 11.0 mg to 13.0 mg, 13.0 mg to 15.0 mg, 15.0 mg to 17.0 mg, 17.0 mg to 19.0 mg, 2.0 mg, 3.0 mg, 4.0 mg, 5.0 mg, 10.0 mg, or 20.0 mg, of zeaxanthin or an equivalent amount of a prodrug, a pharmaceutically acceptable salt, and/or a pharmaceutically acceptable salt of a prodrug of zeaxanthin. In some embodiments, the composition further comprises 2.0 mg of zeaxanthin. In some embodiments, the composition further comprises about 1 mg to about 20 mg of zeaxanthin or an equivalent amount of a prodrug, a pharmaceutically acceptable salt, and/or a pharmaceutically acceptable salt of a prodrug of zeaxanthin. In some embodiments, the composition further comprises about 1 mg to about 20 mg, such as about 1 mg to about 3 mg, about 3 mg to about 5 mg, about 5 mg to about 7 mg, about 7 mg to about 9 mg, about 9 mg to about 11 mg, about 11 mg to about 13 mg, about 13 mg to about 15 mg, about 15 mg to about 17 mg, about 17 mg to about 19 mg, about 2 mg, about 3 mg, about 4 mg, about 5 mg, about 10 mg, or about 20 mg, of zeaxanthin or an equivalent amount of a prodrug, a pharmaceutically acceptable salt, and/or a pharmaceutically acceptable salt of a prodrug of zeaxanthin. In some embodiments, the composition further comprises about 2 mg of zeaxanthin.

In some embodiments, the composition further comprises 350.0 mg to 800.0 mg of vitamin C or an equivalent amount of a prodrug, a pharmaceutically acceptable salt, and/or a pharmaceutically acceptable salt of a prodrug of vitamin C;

50.0 mg to 550.0 mg of vitamin E or an equivalent amount of a prodrug, a pharmaceutically acceptable salt, and/or a pharmaceutically acceptable salt of a prodrug of vitamin E;

20.0 mg to 90.0 mg of zinc or an equivalent amount of a prodrug, a pharmaceutically acceptable form, and/or a pharmaceutically acceptable salt of a prodrug of zinc;

1.5 mg to 2.5 mg of copper or an equivalent amount of a prodrug, a pharmaceutically acceptable form, and/or a pharmaceutically acceptable salt of a prodrug of copper;

0.1 mg to 25 mg of thiamin or an equivalent amount of a prodrug, a pharmaceutically acceptable salt, and/or a pharmaceutically acceptable salt of a prodrug of vitamin B1;

0.1 mg to 25 mg of riboflavin or an equivalent amount of a prodrug, a pharmaceutically acceptable salt, and/or a pharmaceutically acceptable salt of a prodrug of vitamin B2;

1.0 mg to 300.0 mg of nicotinic acid and/or niacinamide or an equivalent amount of a prodrug, a pharmaceutically acceptable salt, and/or a pharmaceutically acceptable salt of a prodrug of vitamin B3;

1.0 mg to 250.0 mg of pantothenic acid or an equivalent amount of a prodrug, a pharmaceutically acceptable salt, and/or a pharmaceutically acceptable salt of a prodrug of vitamin B5;

25.0 mg to 200.0 mg of pyridoxine or an equivalent amount of a prodrug, a pharmaceutically acceptable salt, and/or a pharmaceutically acceptable salt of a prodrug of vitamin B6;

0.01 mg to 1.0 mg of biotin or an equivalent amount of a prodrug, a pharmaceutically acceptable salt, and/or a pharmaceutically acceptable salt of a prodrug of vitamin B7;

0.1 mg to 50 mg of folic acid and/or methyl tetrahydrofolate or an equivalent amount of a prodrug, a pharmaceutically acceptable salt, and/or a pharmaceutically acceptable salt of a prodrug of vitamin B9; and

0.25 mg to 3.0 mg of cyanocobalamin or an equivalent amount of a prodrug, a pharmaceutically acceptable salt, and/or a pharmaceutically acceptable salt of a prodrug of vitamin B12.

In some embodiments, the compositions further comprise 5.0 mg to 50.0 mg, such as 5.0 mg to 15.0 mg, of lutein or an equivalent amount of a prodrug, a pharmaceutically acceptable salt, and/or a pharmaceutically acceptable salt of a prodrug of lutein; and/or 1.0 mg to 2.5 mg, such as 1.5 mg to 2.5 mg, of zeaxanthin or an equivalent amount of a prodrug, a pharmaceutically acceptable salt, and/or a pharmaceutically acceptable salt of a prodrug of zeaxanthin.

In some embodiments, the composition further comprises:

500.0 mg vitamin C, optionally in the form of ascorbic acid;

180.0 mg vitamin E optionally in the form of dl-alpha tocopheryl acetate;

80.0 mg zinc, optionally in the form of zinc oxide;

2.0 mg copper, optionally in the form of copper oxide;

1.5 mg thiamin;

1.7 mg riboflavin;

20.0 mg nicotinic acid and/or niacinamide;

10.0 mg pantothenic acid;

50.0 mg pyridoxine;

0.03 mg biotin;

2.5 mg folic acid and/or methyl tetrahydrofolate; and

1.0 mg cyanocobalamin.

In some embodiments, the composition further comprises 10.0 mg R′,R′ lutein and/or 2.0 mg R′,R′ zeaxanthin.

In some embodiments, the composition further comprises:

500.0 mg vitamin C, optionally in the form of ascorbic acid;

180.0 mg vitamin E optionally in the form of dl-alpha tocopheryl acetate;

80.0 mg zinc, optionally in the form of zinc oxide;

2.0 mg copper, optionally in the form of copper oxide;

3.0 mg thiamin;

3.4 mg riboflavin;

40.0 mg nicotinic acid and/or niacinamide;

20.0 mg pantothenic acid;

50.0 mg pyridoxine;

0.06 mg biotin;

2.5 mg folic acid and/or methyl tetrahydrofolate; and

1.0 mg cyanocobalamin.

In some embodiments, the composition further comprises 10.0 mg R′,R′ lutein and/or 2.0 mg R′,R′ zeaxanthin.

In some embodiments, the composition further comprises:

500.0 mg vitamin C, optionally in the form of ascorbic acid;

180.0 mg vitamin E optionally in the form of dl-alpha tocopheryl acetate;

80.0 mg zinc, optionally in the form of zinc oxide;

2.0 mg copper, optionally in the form of copper oxide;

4.5 mg thiamin;

5.1 mg riboflavin;

60.0 mg nicotinic acid and/or niacinamide;

30.0 mg pantothenic acid;

50.0 mg pyridoxine;

0.09 mg biotin;

2.5 mg folic acid and/or methyl tetrahydrofolate; and

1.0 mg cyanocobalamin.

In some embodiments, the composition further comprises 10.0 mg R′,R′ lutein and/or 2.0 mg R′,R′ zeaxanthin.

In some embodiments, the composition further comprises:

500.0 mg vitamin C, optionally in the form of ascorbic acid;

180.0 mg vitamin E optionally in the form of dl-alpha tocopheryl acetate;

25.0 mg zinc, optionally in the form of zinc oxide;

2.0 mg copper, optionally in the form of copper oxide;

1.5 mg thiamin;

1.7 mg riboflavin;

20.0 mg nicotinic acid and/or niacinamide;

10.0 mg pantothenic acid;

50.0 mg pyridoxine;

0.03 mg biotin;

2.5 mg folic acid and/or methyl tetrahydrofolate; and

1.0 mg cyanocobalamin.

In some embodiments, the composition further comprises 10.0 mg R′,R′ lutein and/or 2.0 mg R′,R′ zeaxanthin.

In some embodiments, the composition further comprises:

500.0 mg vitamin C, optionally in the form of ascorbic acid;

180.0 mg vitamin E optionally in the form of dl-alpha tocopheryl acetate;

25.0 mg zinc, optionally in the form of zinc oxide;

2.0 mg copper, optionally in the form of copper oxide;

3.0 mg thiamin;

3.4 mg riboflavin;

40.0 mg nicotinic acid and/or niacinamide;

20.0 mg pantothenic acid;

50.0 mg pyridoxine;

0.06 mg biotin;

2.5 mg folic acid and/or methyl tetrahydrofolate; and

1.0 mg cyanocobalamin.

In some embodiments, the composition further comprises 10.0 mg R′,R′ lutein and/or 2.0 mg R′,R′ zeaxanthin.

In some embodiments, the composition further comprises:

500.0 mg vitamin C, optionally in the form of ascorbic acid;

180.0 mg vitamin E, optionally in the form of dl-alpha tocopheryl acetate;

25.0 mg zinc, optionally in the form of zinc oxide;

2.0 mg copper;

4.5 mg thiamin;

5.1 mg riboflavin;

60.0 mg nicotinic acid and/or niacinamide;

30.0 mg pantothenic acid;

50.0 mg pyridoxine;

0.09 mg biotin;

2.5 mg folic acid and/or methyl tetrahydrofolate; and

1.0 mg cyanocobalamin.

In some embodiments, the composition further comprises 10.0 mg R′,R′ lutein and/or 2.0 mg R′,R′ zeaxanthin.

In some embodiments, the composition further comprises

about 350 mg to about 800 mg of vitamin C or an equivalent amount of a prodrug, a pharmaceutically acceptable salt, and/or a pharmaceutically acceptable salt of a prodrug of vitamin C;

about 50 mg to about 550 mg of vitamin E or an equivalent amount of a prodrug, a pharmaceutically acceptable salt, and/or a pharmaceutically acceptable salt of a prodrug of vitamin E;

about 20 mg to about 90 mg of zinc or an equivalent amount of a prodrug, a pharmaceutically acceptable form, and/or a pharmaceutically acceptable salt of a prodrug of zinc;

about 1.5 mg to about 2.5 mg of copper or an equivalent amount of a prodrug, a pharmaceutically acceptable form, and/or a pharmaceutically acceptable salt of a prodrug of copper;

about 0.1 mg to about 25 mg of thiamin or an equivalent amount of a prodrug, a pharmaceutically acceptable salt, and/or a pharmaceutically acceptable salt of a prodrug of vitamin B1;

about 0.1 mg to about 25 mg of riboflavin or an equivalent amount of a prodrug, a pharmaceutically acceptable salt, and/or a pharmaceutically acceptable salt of a prodrug of vitamin B2;

about 1 mg to about 300 mg of nicotinic acid and/or niacinamide or an equivalent amount of a prodrug, a pharmaceutically acceptable salt, and/or a pharmaceutically acceptable salt of a prodrug of vitamin B3;

about 1 mg to about 250 mg of pantothenic acid or an equivalent amount of a prodrug, a pharmaceutically acceptable salt, and/or a pharmaceutically acceptable salt of a prodrug of vitamin B5;

about 25 mg to about 200 mg of pyridoxine or an equivalent amount of a prodrug, a pharmaceutically acceptable salt, and/or a pharmaceutically acceptable salt of a prodrug of vitamin B6;

about 0.01 mg to about 1 mg of biotin or an equivalent amount of a prodrug, a pharmaceutically acceptable salt, and/or a pharmaceutically acceptable salt of a prodrug of vitamin B7;

about 0.1 mg to about 50 mg of folic acid and/or methyl tetrahydrofolate or an equivalent amount of a prodrug, a pharmaceutically acceptable salt, and/or a pharmaceutically acceptable salt of a prodrug of vitamin B9; and about 0.25 mg to about 3 mg of cyanocobalamin or an equivalent amount of a prodrug, a pharmaceutically acceptable salt, and/or a pharmaceutically acceptable salt of a prodrug of vitamin B12.

In some embodiments, the compositions further comprise about 5 mg to about 50 mg, such as about 5 mg to about 50 mg, of lutein or an equivalent amount of a prodrug, a pharmaceutically acceptable salt, and/or a pharmaceutically acceptable salt of a prodrug of lutein; and/or about 1 mg to about 20 mg of zeaxanthin or an equivalent amount of a prodrug, a pharmaceutically acceptable salt, and/or a pharmaceutically acceptable salt of a prodrug of zeaxanthin.

In some embodiments, the composition further comprises:

about 500 mg vitamin C, optionally in the form of ascorbic acid;

about 180 mg vitamin E, optionally in the form of dl-alpha tocopheryl acetate;

about 80 mg zinc, optionally in the form of zinc oxide;

about 2 mg copper, optionally in the form of copper oxide;

about 1.5 mg thiamin;

about 1.7 mg riboflavin;

about 20 mg nicotinic acid and/or niacinamide;

about 10 mg pantothenic acid;

about 50 mg pyridoxine;

about 0.03 mg biotin;

about 2.5 mg folic acid and/or methyl tetrahydrofolate; and

about 1 mg cyanocobalamin.

In some embodiments, the composition further comprises about 10 mg R′,R′ lutein and/or about 2 mg R′,R′ zeaxanthin.

In some embodiments, the composition further comprises:

about 500 mg vitamin C, optionally in the form of ascorbic acid;

about 180 mg vitamin E, optionally in the form of dl-alpha tocopheryl acetate;

about 80 mg zinc, optionally in the form of zinc oxide;

about 2 mg copper, optionally in the form of copper oxide;

about 3 mg thiamin;

about 3.4 mg riboflavin;

about 40 mg nicotinic acid and/or niacinamide;

about 20 mg pantothenic acid;

about 50 mg pyridoxine;

about 0.06 mg biotin;

about 2.5 mg folic acid and/or methyl tetrahydrofolate; and

about 1 mg cyanocobalamin.

In some embodiments, the composition further comprises about 10 mg R′,R′ lutein and/or about 2 mg R′,R′ zeaxanthin.

In some embodiments, the composition further comprises:

about 500 mg vitamin C, optionally in the form of ascorbic acid;

about 180 mg vitamin E, optionally in the form of dl-alpha tocopheryl acetate;

about 80 mg zinc, optionally in the form of zinc oxide;

about 2 mg copper, optionally in the form of copper oxide;

about 4.5 mg thiamin;

about 5.1 mg riboflavin;

about 60 mg nicotinic acid and/or niacinamide;

about 30 mg pantothenic acid;

about 50 mg pyridoxine;

about 0.09 mg biotin;

about 2.5 mg folic acid and/or methyl tetrahydrofolate; and

about 1 mg cyanocobalamin.

In some embodiments, the composition further comprises about 10 mg R′,R′ lutein and/or about 2 mg R′,R′ zeaxanthin.

In some embodiments, the composition further comprises:

about 500 mg vitamin C, optionally in the form of ascorbic acid;

about 180 mg vitamin E, optionally in the form of dl-alpha tocopheryl acetate;

about 25 mg zinc, optionally in the form of zinc oxide;

about 2 mg copper, optionally in the form of copper oxide;

about 1.5 mg thiamin;

about 1.7 mg riboflavin;

about 20 mg nicotinic acid and/or niacinamide;

about 10 mg pantothenic acid;

about 50 mg pyridoxine;

about 0.03 mg biotin;

about 2.5 mg folic acid and/or methyl tetrahydrofolate; and

about 1 mg cyanocobalamin.

In some embodiments, the composition further comprises about 10 mg R′,R′ lutein and/or about 2 mg R′,R′ zeaxanthin.

In some embodiments, the composition further comprises:

about 500 mg vitamin C, optionally in the form of ascorbic acid;

about 180 mg vitamin E, optionally in the form of dl-alpha tocopheryl acetate;

about 25 mg zinc, optionally in the form of zinc oxide;

about 2 mg copper, optionally in the form of copper oxide;

about 3 mg thiamin;

about 3.4 mg riboflavin;

about 40 mg nicotinic acid and/or niacinamide;

about 20 mg pantothenic acid;

about 50 mg pyridoxine;

about 0.06 mg biotin;

about 2.5 mg folic acid and/or methyl tetrahydrofolate; and

about 1 mg cyanocobalamin.

In some embodiments, the composition further comprises about 10 mg R′,R′ lutein and/or about 2 mg R′,R′ zeaxanthin.

In some embodiments, the composition further comprises:

about 500 mg vitamin C, optionally in the form of ascorbic acid;

about 180 mg vitamin E, optionally in the form of dl-alpha tocopheryl acetate;

about 25 mg zinc, optionally in the form of zinc oxide;

about 2 mg copper, optionally in the form of copper oxide;

about 4.5 mg thiamin;

about 5.1 mg riboflavin;

about 60 mg nicotinic acid and/or niacinamide;

about 30 mg pantothenic acid;

about 50 mg pyridoxine;

about 0.09 mg biotin;

about 2.5 mg folic acid and/or methyl tetrahydrofolate; and

about 1 mg cyanocobalamin.

In some embodiments, the composition further comprises about 10 mg R′,R′ lutein and/or about 2 mg R′,R′ zeaxanthin.

In some embodiments, the composition further comprises 350.0 mg to 800.0 mg of vitamin C or an equivalent amount of a prodrug, a pharmaceutically acceptable salt, and/or a pharmaceutically acceptable salt of a prodrug of vitamin C; 50.0 mg to 550.0 mg of vitamin E or an equivalent amount of a prodrug, a pharmaceutically acceptable salt, and/or a pharmaceutically acceptable salt of a prodrug of vitamin E; 20.0 mg to 90.0 mg of zinc or an equivalent amount of a prodrug, a pharmaceutically acceptable form, and/or a pharmaceutically acceptable salt of a prodrug of zinc; 1.5 mg to 2.5 mg of copper or an equivalent amount of a prodrug, a pharmaceutically acceptable form, and/or a pharmaceutically acceptable salt of a prodrug of copper; 5.0 mg to 50.0 mg of lutein or an equivalent amount of a prodrug, a pharmaceutically acceptable salt, and/or a pharmaceutically acceptable salt of a prodrug of lutein; and/or 1.5 mg to 20.0 mg of zeaxanthin or an equivalent amount of a prodrug, a pharmaceutically acceptable salt, and/or a pharmaceutically acceptable salt of a prodrug of zeaxanthin.

In some embodiments, the composition further comprises about 350 mg to about 800 mg of vitamin C or an equivalent amount of a prodrug, a pharmaceutically acceptable salt, and/or a pharmaceutically acceptable salt of a prodrug of vitamin C; about 50 mg to about 550 mg of vitamin E or an equivalent amount of a prodrug, a pharmaceutically acceptable salt, and/or a pharmaceutically acceptable salt of a prodrug of vitamin E; about 20 mg to about 90 mg of zinc or an equivalent amount of a prodrug, a pharmaceutically acceptable form, and/or a pharmaceutically acceptable salt of a prodrug of zinc; about 1.5 mg to about 2.5 mg of copper or an equivalent amount of a prodrug, a pharmaceutically acceptable form, and/or a pharmaceutically acceptable salt of a prodrug of copper; about 5 mg to about 50 mg of lutein or an equivalent amount of a prodrug, a pharmaceutically acceptable salt, and/or a pharmaceutically acceptable salt of a prodrug of lutein; and/or about 1 mg to about 20 mg of zeaxanthin or an equivalent amount of a prodrug, a pharmaceutically acceptable salt, and/or a pharmaceutically acceptable salt of a prodrug of zeaxanthin.

In some embodiments, the composition comprises

350.0 mg to 800.0 mg of vitamin C or an equivalent amount of a prodrug, a pharmaceutically acceptable salt, and/or a pharmaceutically acceptable salt of a prodrug of vitamin C;

50.0 mg to 550.0 mg of vitamin E or an equivalent amount of a prodrug, a pharmaceutically acceptable salt, and/or a pharmaceutically acceptable salt of a prodrug of vitamin E;

20.0 mg to 90.0 mg of zinc or an equivalent amount of a prodrug, a pharmaceutically acceptable form, and/or a pharmaceutically acceptable salt of a prodrug of zinc;

1.5 mg to 2.5 mg of copper or an equivalent amount of a prodrug, a pharmaceutically acceptable form, and/or a pharmaceutically acceptable salt of a prodrug of copper;

150.0 mg to 250.0 mg of docosahexanoic acid or an equivalent amount of a prodrug, a pharmaceutically acceptable salt, and/or a pharmaceutically acceptable salt of a prodrug of docosahexanoic acid;

35.0 mg to 45.0 mg of at least one C₂₆₊ acid (5n-3) or an equivalent amount of prodrugs, pharmaceutically acceptable salts, and/or pharmaceutically acceptable salts of prodrugs of C₂₆₊ acids (5n-3); and

35.0 mg to 45.0 mg of eicosapentanoic acid or an equivalent amount of a prodrug, a pharmaceutically acceptable salt, and/or a pharmaceutically acceptable salt of a prodrug of eicosapentanoic acid. In some embodiments, the compositions further comprise 5.0 mg to 50.0 mg of lutein or an equivalent amount of a prodrug, a pharmaceutically acceptable salt, and/or a pharmaceutically acceptable salt of a prodrug of lutein; and/or 1.0 mg to 20.0 mg of zeaxanthin or an equivalent amount of a prodrug, a pharmaceutically acceptable salt, and/or a pharmaceutically acceptable salt of a prodrug of zeaxanthin.

In some embodiments, the composition comprises

40.0 mg at least one C₂₆₊ acid (5n-3);

500.0 mg vitamin C, optionally in the form of ascorbic acid;

180.0 mg vitamin E, optionally in the form of dl-alpha tocopheryl acetate;

80.0 mg zinc, optionally in the form of zinc oxide;

2.0 mg copper, optionally in the form of copper oxide;

200.0 mg docosahexanoic acid; and

40.0 mg eicosapentanoic acid.

In some embodiments, the composition comprises

40.0 mg at least one C₂₆₊ acid (5n-3);

500.0 mg vitamin C, optionally in the form of ascorbic acid;

180.0 mg vitamin E, optionally in the form of dl-alpha tocopheryl acetate;

80.0 mg zinc, optionally in the form of zinc oxide;

2.0 mg copper, optionally in the form of copper oxide;

optionally from 1.0 mg to 200.0 mg C₃₂ acid (6n-3);

200.0 mg docosahexanoic acid; and

40.0 mg eicosapentanoic acid.

In some embodiments, the composition further comprises 10.0 mg R′,R′ lutein and/or 2.0 mg R′,R′ zeaxanthin.

In some embodiments, the composition comprises

about 0.5 mg to about 25 mg of at least one C₂₆₊ acid (5n-3) or an equivalent amount of prodrugs, pharmaceutically acceptable salts, and/or pharmaceutically acceptable salts of prodrugs of C₂₆₊ acids (5n-3);

about 350 mg to about 800 mg of vitamin C or an equivalent amount of a prodrug, a pharmaceutically acceptable salt, and/or a pharmaceutically acceptable salt of a prodrug of vitamin C;

about 50 mg to about 550 mg of vitamin E or an equivalent amount of a prodrug, a pharmaceutically acceptable salt, and/or a pharmaceutically acceptable salt of a prodrug of vitamin E;

about 20 mg to about 90 mg of zinc or an equivalent amount of a prodrug, a pharmaceutically acceptable form, and/or a pharmaceutically acceptable salt of a prodrug of zinc;

about 1.5 mg to about 2.5 mg of copper or an equivalent amount of a prodrug, a pharmaceutically acceptable form, and/or a pharmaceutically acceptable salt of a prodrug of copper;

about 150 mg to about 250 mg of docosahexanoic acid or an equivalent amount of a prodrug, a pharmaceutically acceptable salt, and/or a pharmaceutically acceptable salt of a prodrug of docosahexanoic acid;

about 35 mg to about 45 mg of eicosapentanoic acid or an equivalent amount of a prodrug, a pharmaceutically acceptable salt, and/or a pharmaceutically acceptable salt of a prodrug of eicosapentanoic acid; and. In some embodiments, the compositions further comprise about 5 mg to about 50 mg of lutein or an equivalent amount of a prodrug, a pharmaceutically acceptable salt, and/or a pharmaceutically acceptable salt of a prodrug of lutein; and/or about 1 mg to about 20 mg of zeaxanthin or an equivalent amount of a prodrug, a pharmaceutically acceptable salt, and/or a pharmaceutically acceptable salt of a prodrug of zeaxanthin.

In some embodiments, the composition comprises

0.5 mg to 50 mg of at least one C₂₆₊ acid (5n-3) or an equivalent amount of prodrugs, pharmaceutically acceptable salts, and/or pharmaceutically acceptable salts of prodrugs of C₂₆₊ acids (5n-3);

250 mg vitamin C or an equivalent amount of a prodrug, a pharmaceutically acceptable form, and/or a pharmaceutically acceptable salt of a prodrug of vitamin C;

90 mg vitamin E or an equivalent amount of a prodrug, a pharmaceutically acceptable form, and/or a pharmaceutically acceptable salt of a prodrug of vitamin E;

40 mg zinc or an equivalent amount of a prodrug, a pharmaceutically acceptable salt, and/or a pharmaceutically acceptable salt of a prodrug of zinc;

1 mg copper or an equivalent amount of a prodrug, a pharmaceutically acceptable salts, and/or a pharmaceutically acceptable salt of a prodrug of copper;

5 mg lutein, prodrugs thereof, and pharmaceutically acceptable salts of any of the foregoing; and 1 mg zeaxanthin, prodrugs thereof, and pharmaceutically acceptable salts of any of the foregoing.

In some embodiments, the composition comprises

0.5 mg to 50 mg of at least one C₂₆₊ acid (5n-3) or an equivalent amount of prodrugs, pharmaceutically acceptable salts, and/or pharmaceutically acceptable salts of prodrugs of C₂₆₊ acids (5n-3);

500 mg vitamin C or an equivalent amount of a prodrug, a pharmaceutically acceptable form, and/or a pharmaceutically acceptable salt of a prodrug of vitamin C;

180 mg vitamin E or an equivalent amount of a prodrug, a pharmaceutically acceptable form, and/or a pharmaceutically acceptable salt of a prodrug of vitamin E;

80 mg zinc or an equivalent amount of a prodrug, a pharmaceutically acceptable salt, and/or a pharmaceutically acceptable salt of a prodrug of zinc;

2 mg copper or an equivalent amount of a prodrug, a pharmaceutically acceptable salts, and/or a pharmaceutically acceptable salt of a prodrug of copper;

10 mg lutein, prodrugs thereof, and pharmaceutically acceptable salts of any of the foregoing; and

2 mg zeaxanthin, prodrugs thereof, and pharmaceutically acceptable salts of any of the foregoing.

In some embodiments, the composition comprises

about 500 mg vitamin C, optionally in the form of ascorbic acid;

about 180 mg vitamin E, optionally in the form of dl-alpha tocopheryl acetate;

about 80 mg zinc, optionally in the form of zinc oxide;

about 2 mg copper, optionally in the form of copper oxide;

about 200 mg docosahexanoic acid;

about 40 mg at least one C₂₆₊ acid (5n-3); and

about 40 mg eicosapentanoic acid.

In some embodiments, the composition further comprises about 10 mg R′,R′ lutein and/or about 2 mg R′,R′ zeaxanthin.

The compositions disclosed herein may be prepared according to any known method for the manufacture of dietary supplements or pharmaceutical preparations. As will be appreciated by those of ordinary skill in the art, a number of methods are known. For information concerning materials, equipment and processes for preparing formulations and dosage forms, see “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et. al., (Media, Pa.: Williams and Wilkins, 1995) which provide information on carriers, materials, equipment and process for preparing formulations.

A method of manufacturing the compositions disclosed herein may comprise combining the recited vitamins as well as any desired excipients and mechanically mixing, such as for example, using a blender to form a blend. If necessary, the blend may be then tumbled until uniform. The blend may be then compressed using a tablet press to form tablets. Optionally a coating may be sprayed on the tablets and the tablets tumbled until dry. Alternatively, the blend may be placed in medium chain triglycerides to form a slurry for containment in a soft gel capsule, the blend may be placed in a gelatin capsule or the blend may be placed in other dosage forms known to those skilled in the art.

Methods for treating and/or preventing at least one disease, disorder, and/or condition associated with a loss of visual acuity including, for example, age-related macular degeneration (AMD), are disclosed herein, the methods comprising administering to a subject a composition disclosed herein.

The at least one disease, disorder, and/or condition associated with a loss of visual acuity includes age-related macular degeneration (AMD). In some embodiments, the method disclosed herein is effective to prevent, attenuate, or inhibit the progression of AMD. In some embodiments, AMD is wet AMD or dry AMD.

In some embodiments, the at least one disease, disorder, and/or condition associated with a loss of visual acuity is chosen from macular degeneration, age-related macular degeneration (AMD), atrophy of retinal pigmented epithelium (RPE), atrophy of at least one photoreceptor, drusen, drusenoid pigment epithelial detachment (PED), diabetic retinopathy, cataracts, retinitis pigmentosa, glaucoma, choroidal neovascularization, retinal degeneration, Stargardts disease, and oxygen-induced retinopathy.

In some embodiments of the methods disclosed herein, the compositions disclosed herein are administered to a subject in one, two, three, or four doses daily. In some embodiments, the compositions are each administered in the form of one, two, three, or four dosage units one, two, three, or four times daily, such as in the form of two tablets taken twice daily or in the form of one tablet taken twice daily.

Also disclosed herein are methods of regressing drusen and/or drusenoid pigment epithelial detachment (PED) comprising administering to a subject a composition disclosed herein. Methods for treating and/or preventing atrophy of retinal pigmented epithelium (RPE) and/or at least one photoreceptor are disclosed herein, wherein the methods comprise administering to a subject a composition disclosed herein. Methods for treating and/or preventing vision loss and/or improving acuity also disclosed herein.

In some embodiments, administration of a composition as described herein causes a complete disappearance (i.e., 100% regression) of the drusen. In some embodiments, administration of a composition as described herein prevents atrophy of the RPE and/or photoreceptors in a patient (i.e., geographic atrophy).

In some embodiments of the disclosed methods, the subjects to whom the compositions are administered are chosen from subjects suspected of suffering from at least one disease, disorder, and/or condition associated with a loss of visual acuity, subjects known to be suffering from at least one disease, disorder, and/or condition associated with a loss of visual acuity, and subjects at risk of developing at least one disease, disorder, and/or condition associated with a loss of visual acuity.

In some embodiments, the disclosed methods further comprise identifying a subject as being at risk of developing at least one disease, disorder, and/or condition associated with a loss of visual acuity. In some embodiments, the disclosed methods further comprise identifying a subject as being at risk of developing AMD. In some embodiments, the disclosed methods further comprise identifying a subject as having AMD. In some embodiments, the disclosed methods further comprise identifying a subject as being at risk for AMD progression.

In some embodiments of the methods described herein, the subject is a human. In some embodiments of the methods described herein, the subject is a non-human animal. Non-human animals include mammals, for example, non-human primates, swine, equine, canine, feline, bovine, rodents, and other domestic, farm, and zoo animals.

A subject at risk of developing at least one disease, disorder, and/or condition associated with a loss of visual acuity can be identified by one or more diagnostic or prognostic assays described herein and/or known to those of ordinary skill in the art.

In some embodiments, the methods disclosed herein treat and/or prevent at least one disease, disorder, and/or condition associated with a loss of visual acuity as evidenced by an improvement of visual acuity. In some embodiments, the methods disclosed herein further comprise monitoring the subject for efficacy of administering to the subject a composition as disclosed herein. For example, in some embodiments, the methods disclosed herein further comprise monitoring a subject for improvement of visual acuity which comprises measuring a parameter indicative of visual acuity in the subject at a first time point prior to administration of the composition disclosed herein, measuring the same parameter in the subject at a second time point after administration of the composition disclosed herein, and comparing the two measurements to assess improvement.

The methods disclosed herein comprise administration of a daily dose of a composition disclosed herein. Determining and adjusting an appropriate dosing regimen (e.g., adjusting the number of doses and frequency of dosing) per day can be performed by one of ordinary skill in the relevant art, and will depend upon various factors such as the nature and progression of at least one disease, disorder, and/or condition associated with a loss of visual acuity, and the health and/or age of the subject.

In some embodiments, the composition disclosed herein is formulated in the form of one tablet, two tablets, three tablets, four tablets, etc. and is administered to a subject in a single dose per day, in two doses per day, in three doses per day, in four doses per day, etc. or up to, for example, ten doses per day. In some embodiments, the composition disclosed herein is formulated in the form of four tablets which are administered to a subject in two doses of two tablets each per day. In some embodiments, the composition disclosed herein is administered to a subject for at least one week, at least two weeks, at least three weeks, at least one month, at least 2 months, at least 3 months, at least 4 months, at least 5 months, at least 6 months, at least 12 months, at least one year, or for more than one year.

Also disclosed herein are methods of preventing, stabilizing, reversing and/or treating macular degeneration or visual acuity loss by reducing the risk of developing late stage or advanced age-related macular degeneration in persons with early age-related macular degeneration comprising providing a subject a daily dosage of not less than about 1.5 mg thiamin, about 1.7 mg riboflavin, about 20 mg nicotinic acid and/or niacinamide, about 10 mg pantothenic acid, and about 50 mg pyridoxine. In some embodiments, the daily dosage is provided in the form of two tablets taken twice daily or in the form of one tablet taken twice daily.

The effectiveness of the compositions and methods of the present disclosure, for example, in treating and/or preventing at least one disease, disorder, and/or condition associated with a loss of visual acuity, can be determined by a person of ordinary skill in the relevant art. One or any combination of diagnostic methods, including physical examination, assessment and monitoring of symptoms, and performance of analytical tests and methods described herein, may be used for monitoring the at least one disease, disorder, and/or condition. For example, ocular examinations such as biomicroscopy, tonometry, stereoscopic fundus examination (e.g., color fundus photography), macular function assessment, optical coherence tomography (OCT), autofluorescence, and/or angiography (e.g., fluorescein angiography, and OCT based angiography (OCTA)) may be used.

For example, visual acuity can be assessed by any suitable manner known to one of ordinary skill in the art. In some embodiments, visual acuity is assessed by determining the smallest letters the patient can read on a standard vision chart at a set distance. Further for example, progression of AMD can be evaluated by measuring a parameter, such as neovascularization. Maintenance or a reduction in the measured parameter from the first time point to the second time point is indicative of the prevention of AMD progression. In some embodiments, progression of AMD and/or drusen regression is evaluated by determining a particular parameter of drusen in a patient at a first time point (e.g., prior to administration of a high-dose statin), determining the same parameter in the same patient at a second time point (e.g., after administration of a high-dose statin), and comparing the measured parameter at the first time point and the second time point. The parameter of drusen can be, for example, volume, height, diameter, and/or number. For example, drusen regression can be by at least 5%, at least 10% at least 15%, at 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, drusen are reduced 5% to at least 40%, such as 90%. In addition, atrophy can be evaluated, for example, by measuring a parameter such as autofluorescence or retinal thickness and PED can be evaluated for flattening, for example, by measuring a parameter such as volume, height, and/or diameter, and can be evaluated for re-attaching to the Bruch's membrane by, for example, measuring a parameter such as the distance of the separation.

### EXAMPLE 1: IN VITRO ACTIVITY OF C₃₂ ACID (6N-3)

Three month old wild-type (WT) C57BL/6J mice (22±3 g) were each gavage fed 6 mg per day of C₃₂ acid (6n-3) (“32:6 n-3”) loaded VLCPUFA liposomes (0.1 mL) (250 mg/kg body weight) prepared as disclosed in Gorusupudi et al. PNAS 118(6), e2017739118 (2021), which is incorporated herein by reference in its entirety. The mice were killed 0, 2, 4, 8, and 24 h after gavage (n=4 mice/time interval) to harvest the blood and other tissues. Eyes were dissected under a microscope to separate the retina from the RPE. One pair of retinas/mouse was used for lipid extraction, and blood was centrifuged for 5 minutes at 7,500 rpm to separate serum from RBCs. Serum, RBCs, brain, liver, retina, and RPE were used for lipid extractions and GC-MS analysis.

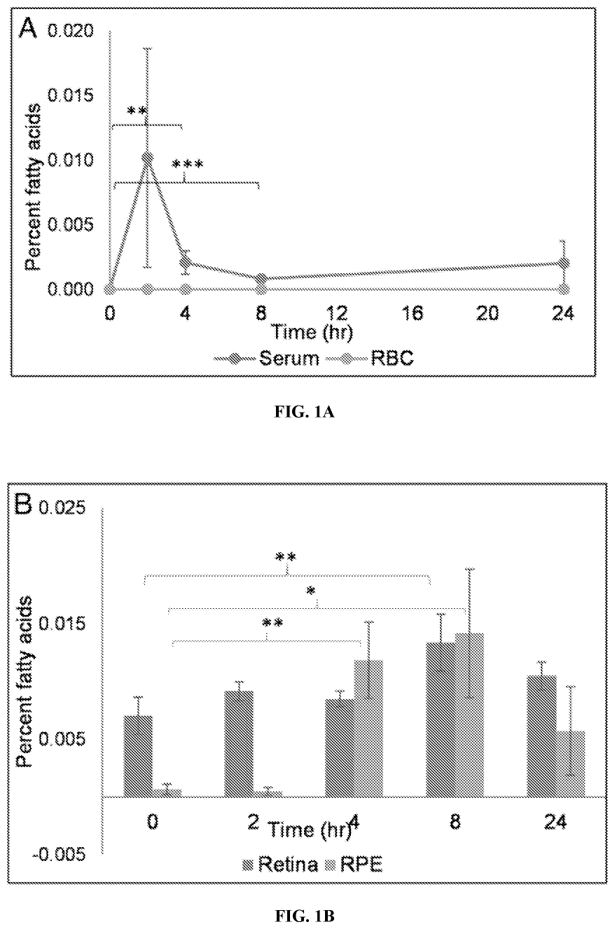

As shown in FIG. 1A, serum 32:6 n-3 became detectable within 2 h after oral administration and then reached a lower steady-state concentration over the next 24 h. A significant increase in 32:6 n-3 in the retina and RPE was observed 4 to 8 h after gavage (FIG. 1B). On the other hand, 32:6 n-3 remained undetectable in liver, brain, and red blood cell (RBC) membranes throughout the 24 h period. Longer-term absorption was studied by gavage feeding VLCPUFA-loaded liposomes (0.1 mL) to WT (22±3 g) and conditional KO mice developed with an intention to ablate Elov14 expression completely in both rods and cones (E4cKO mice) (23±3 g) for 15 d with 0, 1, and 2 mg/d 32:6 n-3 (n=6 mice/group). Mice were fed each morning with liposomes prepared as above containing 0 mg/d (0 mg/kg BW), 1 mg/d (40 mg/kg BW), or 2 mg/d (80 mg/kg BW) of 32:6 n-3. Mice were killed at the end of the experiments, and retina, RPE, liver, serum, RBCs, and brain tissues were harvested, extracted for lipids, and analyzed by GC-MS.

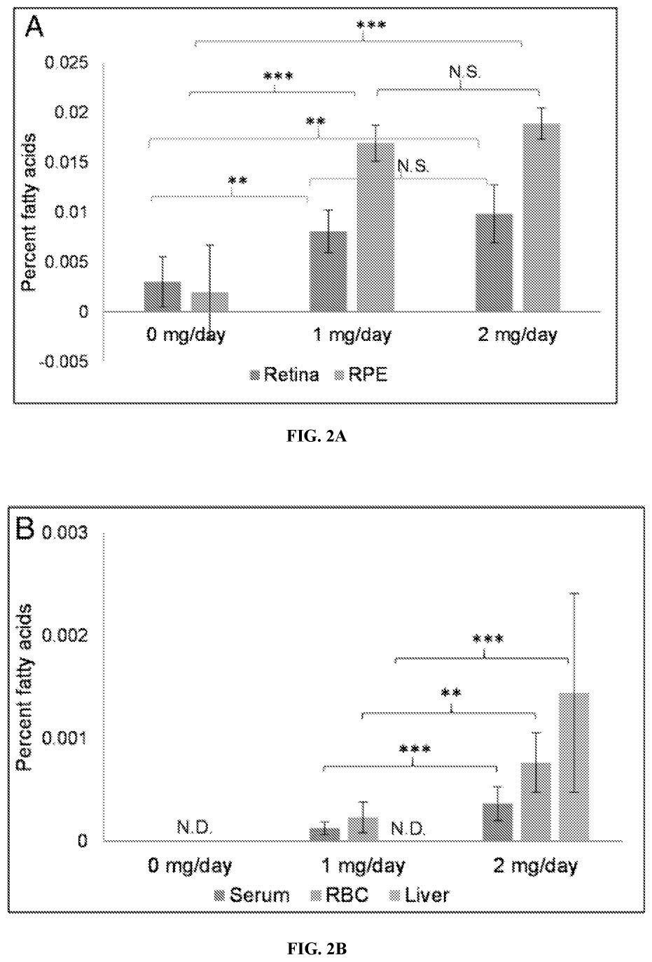

As shown in FIG. 2A, C₃₂ acid (6n-3) levels significantly increased by more than twofold in the retina and sevenfold in RPE in both 32:6 n-3-fed groups in comparison to 0 mg/d controls. A significant increase (P<0.05) in total retinal VLCPUFAs was also observed in the 2 mg/d group (0.872±0.10%) in comparison to the 1 mg/d (0.535±0.06%) and 0 mg/d (0.531±0.16%) groups. Serum and RBC VLCPUFAs remained undetectable in the control mice and significantly increased in the 32:6 n-3-fed groups, as shown in FIG. 2B. The liver had detectable levels of 32:6 n-3 only in the 2 mg/d group (FIG. 2B), while no VLCPUFAs were ever detectable in the brain.

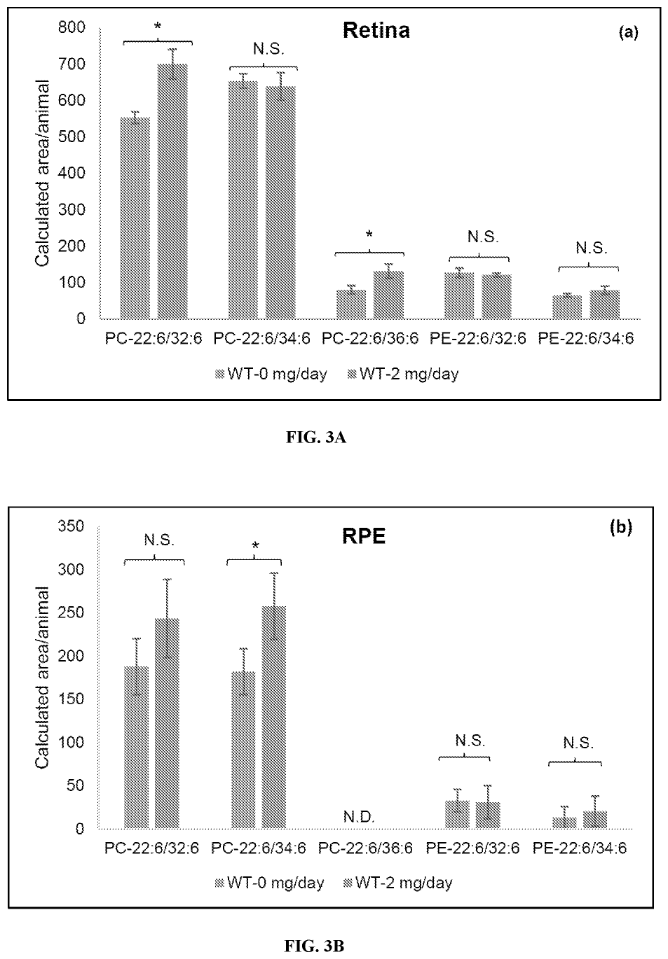

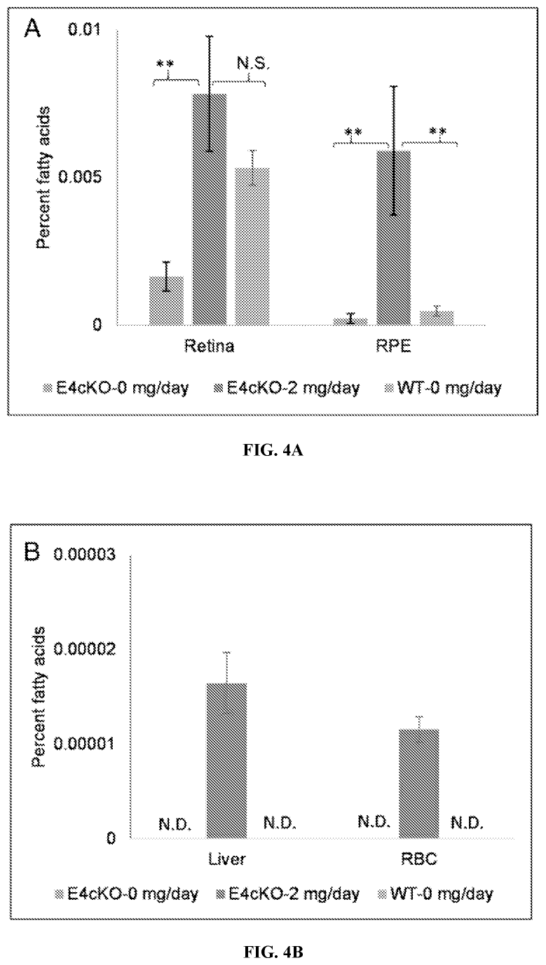

Retina and RPE were extracted for phospholipid analysis using liquid chromatography-tandem MS (LC-MS/MS), and increases in phosphatidylcholine (PC)-22:6/32:6 were detected in the retina and RPE in the supplemented WT animals (FIGS. 3A and 3B). Retinal and RPE 32:6 n-3 levels in the E4cKO mice were one-third and one-half of the levels in the age-matched WT mice, respectively (FIG. 4A). After 15 d of gavage feeding with 2 mg/d of synthetic 32:6 n-3, the 8-mo-old E4cKO mice had a nearly fivefold increase in their 32:6 n-3 levels in the retina (P<0.01) and a 27-fold increase in the RPE (P=0.001) relative to control-fed E4cKO mice (FIG. 4A). There was a significant increase (P<0.05) in total retinal VLCPUFAs in the E4cKO mice fed 2 mg/d 32:6 n-3 (0.088±0.02%) in comparison to the 0 mg/d E4cKO mice (0.030±0.01%) that approached the levels in age-matched 0 mg/d WT mice (0.102±0.008%). Liver and RBC 32:6 n-3 became detectable in the mice fed 2 mg/d 32:6 n-3 relative to the undetectable levels in the 0 mg/d E4cKO and WT mice (FIG. 4A).

These findings support the inventor's belief that compositions comprising at least one very long chain fatty acid chosen from C₂₆₊ acids (5n-3), prodrugs thereof, and pharmaceutically acceptable salts of any of the foregoing may be used to maintain normal physiology of the retina and also may be used to improve visual acuity. Thus, the findings support the presently claimed uses of the compositions comprising at least one very long chain fatty acid chosen from C₂₆₊ acids (5n-3), prodrugs thereof, and pharmaceutically acceptable salts of any of the foregoing, including methods of delaying progression and/or onset of macular degeneration and methods of treating and/or preventing at least one disease, disorder, and/or condition associated with a loss of visual acuity.

### Prophetic Examples

The following compositions can be prepared. The amounts in each composition are set forth on a daily dosage basis. Each composition can be formulated into two or more dosage units, which dosage units can be administered to a subject daily. Each active ingredient in each composition can be in the form recited (e.g., acid), a prodrug thereof, and/or a pharmaceutically acceptable salt of the recited form and/or a prodrug thereof.

| | |
|---|---|
| Active Ingredient | Amount (mg) |

| | | | | | | |
|---|---|---|---|---|---|---|
| C₃₄ acid (5n-3) | 1 | 10 | 25 | 100 | 500 | 1000 |

| | |
|---|---|
| Active Ingredient | Amount (mg) |

| | | | | | | |
|---|---|---|---|---|---|---|
| C₃₀ acid (5n-3) | 1 | 10 | 25 | 100 | 500 | 1000 |

| | |
|---|---|
| Active Ingredient | Amount (mg) |

| | | | | | | |
|---|---|---|---|---|---|---|
| C₂₆ acid (5n-3) | 1 | 10 | 25 | 100 | 500 | 1000 |

| Active Ingredient | Amount (mg) | | | | | |
|---|---|---|---|---|---|---|
| C₃₄ acid (5n-3) | 0.33 | 3.33 | 8.33 | 33.33 | 166.67 | 333.33 |
| C₃₀ acid (5n-3) | 0.33 | 3.33 | 8.33 | 33.33 | 166.67 | 333.33 |
| C₂₆ acid (5n-3) | 0.33 | 3.33 | 8.33 | 33.33 | 166.67 | 333.33 |

| | |
|---|---|
| Active Ingredient | Amount (mg) |

| | | | | | | |
|---|---|---|---|---|---|---|
| C₃₄ acid (5n-3) | 0.5 | 5 | 12.5 | 50 | 250 | 500 |
| C₃₀ acid (5n-3) | 0.5 | 5 | 12.5 | 50 | 250 | 500 |

| | |
|---|---|
| Active Ingredient | Amount (mg) |

| | | | | | | |
|---|---|---|---|---|---|---|
| C₃₄ acid (5n-3) | 0.1 | 1 | 2.5 | 10 | 50 | 100 |
| C₃₀ acid (5n-3) | 0.9 | 9 | 22.5 | 90 | 450 | 900 |

| | |
|---|---|
| Active Ingredient | Amount (mg) |

| | | | | | | |
|---|---|---|---|---|---|---|
| C₃₄ acid (5n-3) | 0.9 | 9 | 22.5 | 90 | 450 | 900 |
| C₃₀ acid (5n-3) | 0.1 | 1 | 2.5 | 10 | 50 | 100 |

| Active Ingredient | Amount (mg) | | | | | | |
|---|---|---|---|---|---|---|---|
| C₂₆₋₃₄ acid (6n-3) | 10.0 | 24.0 | 420 | 120.0 | 1.2 | 150.0 | 200.0 |
| DHA | 100.0 | 150.0 | 0 | 120.0 | 0 | 100 | 200.0 |
| C₂₆₋₃₄ acid (5n-3) | 2.0 | 6.0 | 84 | 120.0 | 1.2 | 30.0 | 40.0 |
| EPA | 0 | 100.0 | 0 | 120.0 | 0 | 20.0 | 40.0 |

| Active Ingredient | Amount (mg) | | | | | | | | | | | |
|---|---|---|---|---|---|---|---|---|---|---|---|---|
| C₂₆₋₃₄ acid (5n-3) | 84 | 84 | 84 | 40.0 | 40.0 | 40.0 | 30.0 | 30.0 | 30.0 | 120.0 | 120.0 | 120.0 |
| DHA | 0 | 0 | 0 | 200.0 | 200.0 | 200.0 | 100 | 100 | 100 | 120.0 | 120.0 | 120.0 |
| EPA | 0 | 0 | 0 | 40.0 | 40.0 | 40.0 | 20.0 | 20.0 | 20.0 | 120.0 | 120.0 | 120.0 |
| Vitamin B1 (Thiamin) | 1.5 | 3.0 | 4.5 | 1.5 | 3.0 | 4.5 | 1.5 | 3.0 | 4.5 | 1.5 | 3.0 | 4.5 |
| Vitamin B2 (Riboflavin) | 1.7 | 3.4 | 5.1 | 1.7 | 3.4 | 5.1 | 1.7 | 3.4 | 5.1 | 1.7 | 3.4 | 5.1 |
| Vitamin B3 (Nicotinic acid and/or | 20 | 40 | 60 | 20 | 40 | 60 | 20 | 40 | 60 | 20 | 40 | 60 |
| niacinamide) | | | | | | | | | | | | |
| Vitamin B5 (Pantothenic acid) | 10 | 20 | 30 | 10 | 20 | 30 | 10 | 20 | 30 | 10 | 20 | 30 |
| Vitamin B6 (Pyridoxine) | 50 | 50 | 50 | 50 | 50 | 50 | 50 | 50 | 50 | 50 | 50 | 50 |
| Vitamin B7 (Biotin) | 0.03 | 0.06 | 0.09 | 0.03 | 0.06 | 0.09 | 0.03 | 0.06 | 0.09 | 0.03 | 0.06 | 0.09 |
| Vitamin B9 (Folic acid and/or Methyl | 2.5 | 2.5 | 2.5 | 2.5 | 2.5 | 2.5 | 2.5 | 2.5 | 2.5 | 2.5 | 2.5 | 2.5 |
| tetrahydrofolate) | | | | | | | | | | | | |
| Vitamin B12 (Cyanocobalamin) | 1.0 | 1.0 | 1.0 | 1.0 | 1.0 | 1.0 | 1.0 | 1.0 | 1.0 | 1.0 | 1.0 | 1.0 |

| Active Ingredient | Amount (mg) | | | | | | | | | | | |
|---|---|---|---|---|---|---|---|---|---|---|---|---|
| C₂₆₋₃₄ acid (5n-3) | 6.0 | 6.0 | 6.0 | 2.0 | 2.0 | 2.0 | 0.4 | 0.4 | 0.4 | 1.2 | 1.2 | 1.2 |
| DHA | 150.0 | 150.0 | 150.0 | 100.0 | 100.0 | 100.0 | 0 | 0 | 0 | 0 | 0 | 0 |
| EPA | 100.0 | 100.0 | 100.0 | 0 | 0 | 0 | 0 | 0 | 0 | 0 | 0 | 0 |
| Vitamin B1 (Thiamin) | 1.5 | 3.0 | 4.5 | 1.5 | 3.0 | 4.5 | 1.5 | 3.0 | 4.5 | 1.5 | 3.0 | 4.5 |
| Vitamin B2 (Riboflavin) | 1.7 | 3.4 | 5.1 | 1.7 | 3.4 | 5.1 | 1.7 | 3.4 | 5.1 | 1.7 | 3.4 | 5.1 |
| Vitamin B3 (Nicotinic acid and/or niacinamide) | 20 | 40 | 60 | 20 | 40 | 60 | 20 | 40 | 60 | 20 | 40 | 60 |
| Vitamin B5 (Pantothenic acid) | 10 | 20 | 30 | 10 | 20 | 30 | 10 | 20 | 30 | 10 | 20 | 30 |
| Vitamin B6 (Pyridoxine) | 50 | 50 | 50 | 50 | 50 | 50 | 50 | 50 | 50 | 50 | 50 | 50 |
| Vitamin B7 (Biotin) | 0.03 | 0.06 | 0.09 | 0.03 | 0.06 | 0.09 | 0.03 | 0.06 | 0.09 | 0.03 | 0.06 | 0.09 |
| Vitamin B9 (Folic acid and/or Methyl | 2.5 | 2.5 | 2.5 | 2.5 | 2.5 | 2.5 | 2.5 | 2.5 | 2.5 | 2.5 | 2.5 | 2.5 |
| tetrahydrofolate) | | | | | | | | | | | | |
| Vitamin B12 (Cyanocobalamin) | 1.0 | 1.0 | 1.0 | 1.0 | 1.0 | 1.0 | 1.0 | 1.0 | 1.0 | 1.0 | 1.0 | 1.0 |

| Active Ingredient | Amount (mg) | | | | | |
|---|---|---|---|---|---|---|
| C₂₆₋₃₄ acid (5n-3) | 84 | 84 | 84 | 84 | 84 | 84 |
| DHA | 0 | 0 | 0 | 0 | 0 | 0 |
| EPA | 0 | 0 | 0 | 0 | 0 | 0 |
| Vitamin C (Ascorbic acid or equivalent) | 500.0 | 500.0 | 500.0 | 500.0 | 500.0 | 500.0 |
| Vitamin E (alpha tocopherol or equivalent) | 180.0 | 180.0 | 180.0 | 180.0 | 180.0 | 180.0 |
| Zinc (Zinc oxide or equivalent) | 25.0 | 80.0 | 25.0 | 80.0 | 25.0 | 80.0 |
| Copper (Copper oxide or equivalent) | 2.0 | 2.0 | 2.0 | 2.0 | 2.0 | 2.0 |
| Lutein (R′R′) | 10.0 | 10.0 | 10.0 | 10.0 | 10.0 | 10.0 |
| Zeaxanthin (R′R′) | 2.0 | 2.0 | 2.0 | 2.0 | 2.0 | 2.0 |
| Vitamin B1 (Thiamin) | 3.0 | 3.0 | 1.5 | 1.5 | 4.5 | 4.5 |
| Vitamin B2 (Riboflavin) | 3.4 | 3.4 | 1.7 | 1.7 | 5.1 | 5.1 |
| Vitamin B3 (Nicotinic acid and/or niacinamide) | 40.0 | 40.0 | 20.0 | 20.0 | 60.0 | 60.0 |
| Vitamin B5 (Pantothenic acid) | 20.0 | 20.0 | 10.0 | 10.0 | 30.0 | 30.0 |
| Vitamin B6 (Pyridoxine) | 50.0 | 50.0 | 50.0 | 50.0 | 50.0 | 50.0 |
| Vitamin B7 (Biotin) | 0.06 | 0.06 | 0.03 | 0.03 | 0.09 | 0.09 |
| Vitamin B9 (Folic acid and/or Methyl tetrahydrofolate) | 2.5 | 2.5 | 2.5 | 2.5 | 2.5 | 2.5 |
| Vitamin B12 (Cyanocobalamin) | 1.0 | 1.0 | 1.0 | 1.0 | 1.0 | 1.0 |

| Active Ingredient | Amount (mg) | | | | | |
|---|---|---|---|---|---|---|
| C₂₆₋₃₄ acid (5n-3) | 40.0 | 40.0 | 40.0 | 40.0 | 40.0 | 40.0 |
| DHA | 200.0 | 200.0 | 200.0 | 200.0 | 200.0 | 200.0 |
| EPA | 40.0 | 40.0 | 40.0 | 40.0 | 40.0 | 40.0 |
| Vitamin C (Ascorbic acid or equivalent) | 500.0 | 500.0 | 500.0 | 500.0 | 500.0 | 500.0 |
| Vitamin E (alpha tocopherol or equivalent) | 180.0 | 180.0 | 180.0 | 180.0 | 180.0 | 180.0 |
| Zinc (Zinc oxide or equivalent) | 25.0 | 80.0 | 25.0 | 80.0 | 25.0 | 80.0 |
| Copper (Copper oxide or equivalent) | 2.0 | 2.0 | 2.0 | 2.0 | 2.0 | 2.0 |
| Lutein (R′R′) | 10.0 | 10.0 | 10.0 | 10.0 | 10.0 | 10.0 |
| Zeaxanthin (R′R′) | 2.0 | 2.0 | 2.0 | 2.0 | 2.0 | 2.0 |
| Vitamin B1 (Thiamin) | 1.5 | 3.0 | 3.0 | 4.5 | 4.5 | 1.5 |
| Vitamin B2 (Riboflavin) | 1.7 | 3.4 | 3.4 | 5.1 | 5.1 | 1.7 |
| Vitamin B3 (Nicotinic acid and/or niacinamide) | 20.0 | 40.0 | 40.0 | 60.0 | 60.0 | 20.0 |
| Vitamin B5 (Pantothenic acid) | 10.0 | 20.0 | 20.0 | 30.0 | 30.0 | 10.0 |
| Vitamin B6 (Pyridoxine) | 50.0 | 50.0 | 50.0 | 50.0 | 50.0 | 50.0 |
| Vitamin B7 (Biotin) | 0.03 | 0.06 | 0.06 | 0.09 | 0.09 | 0.03 |
| Vitamin B9 (Folic acid and/or Methyl tetrahydrofolate) | 2.5 | 2.5 | 2.5 | 2.5 | 2.5 | 2.5 |
| Vitamin B12 (Cyanocobalamin) | 1.0 | 1.0 | 1.0 | 1.0 | 1.0 | 1.0 |

| Active Ingredient | Amount (mg) | | | | | |
|---|---|---|---|---|---|---|
| C₂₆₋₃₄ acid (5n-3) | 40.0 | 40.0 | 40.0 | 40.0 | 40.0 | 40.0 |
| C₂₆₊ acid (6n-3) | 10.0 | 10.0 | 10.0 | 10.0 | 10.0 | 10.0 |
| DHA | 200.0 | 200.0 | 200.0 | 200.0 | 200.0 | 200.0 |
| EPA | 40.0 | 40.0 | 40.0 | 40.0 | 40.0 | 40.0 |
| Vitamin C (Ascorbic acid or equivalent) | 500.0 | 500.0 | 500.0 | 500.0 | 500.0 | 500.0 |
| Vitamin E (alpha tocopherol or equivalent) | 180.0 | 180.0 | 180.0 | 180.0 | 180.0 | 180.0 |
| Zinc (Zinc oxide or equivalent) | 25.0 | 80.0 | 25.0 | 80.0 | 25.0 | 80.0 |
| Copper (Copper oxide or equivalent) | 2.0 | 2.0 | 2.0 | 2.0 | 2.0 | 2.0 |
| Lutein (R′R′) | 10.0 | 10.0 | 10.0 | 10.0 | 10.0 | 10.0 |
| Zeaxanthin (R′R′) | 2.0 | 2.0 | 2.0 | 2.0 | 2.0 | 2.0 |
| Vitamin B1 (Thiamin) | 1.5 | 3.0 | 3.0 | 4.5 | 4.5 | 1.5 |
| Vitamin B2 (Riboflavin) | 1.7 | 3.4 | 3.4 | 5.1 | 5.1 | 1.7 |
| Vitamin B3 (Nicotinic acid and/or niacinamide) | 20.0 | 40.0 | 40.0 | 60.0 | 60.0 | 20.0 |
| Vitamin B5 (Pantothenic acid) | 10.0 | 20.0 | 20.0 | 30.0 | 30.0 | 10.0 |
| Vitamin B6 (Pyridoxine) | 50.0 | 50.0 | 50.0 | 50.0 | 50.0 | 50.0 |
| Vitamin B7 (Biotin) | 0.03 | 0.06 | 0.06 | 0.09 | 0.09 | 0.03 |
| Vitamin B9 (Folic acid and/or Methyl tetrahydrofolate) | 2.5 | 2.5 | 2.5 | 2.5 | 2.5 | 2.5 |
| Vitamin B12 (Cyanocobalamin) | 1.0 | 1.0 | 1.0 | 1.0 | 1.0 | 1.0 |

| Active Ingredient | Amount (mg) | | | | | |
|---|---|---|---|---|---|---|
| C₂₆₋₃₄ acid (5n-3) | 30.0 | 30.0 | 30.0 | 30.0 | 30.0 | 30.0 |
| DHA | 100 | 100 | 100 | 100 | 100 | 100 |
| EPA | 20.0 | 20.0 | 20.0 | 20.0 | 20.0 | 20.0 |
| Vitamin C (Ascorbic acid or equivalent) | 500.0 | 500.0 | 500.0 | 500.0 | 500.0 | 500.0 |
| Vitamin E (alpha tocopherol or equivalent) | 180.0 | 180.0 | 180.0 | 180.0 | 180.0 | 180.0 |
| Zinc (Zinc oxide or equivalent) | 80.0 | 25.0 | 80.0 | 25.0 | 80.0 | 25.0 |
| Copper (Copper oxide or equivalent) | 2.0 | 2.0 | 2.0 | 2.0 | 2.0 | 2.0 |
| Lutein (R′R′) | 10.0 | 10.0 | 10.0 | 10.0 | 10.0 | 10.0 |
| Zeaxanthin (R′R′) | 2.0 | 2.0 | 2.0 | 2.0 | 2.0 | 2.0 |
| Vitamin B1 (Thiamin) | 4.5 | 3.0 | 3.0 | 1.5 | 1.5 | 4.5 |
| Vitamin B2 (Riboflavin) | 5.1 | 3.4 | 3.4 | 1.7 | 1.7 | 5.1 |
| Vitamin B3 (Nicotinic acid and/or niacinamide) | 60.0 | 40.0 | 40.0 | 20.0 | 20.0 | 60.0 |
| Vitamin B5 (Pantothenic acid) | 30.0 | 20.0 | 20.0 | 10.0 | 10.0 | 30.0 |
| Vitamin B6 (Pyridoxine) | 50.0 | 50.0 | 50.0 | 50.0 | 50.0 | 50.0 |
| Vitamin B7 (Biotin) | 0.09 | 0.06 | 0.06 | 0.03 | 0.03 | 0.09 |
| Vitamin B9 (Folic acid and/or Methyl tetrahydrofolate) | 2.5 | 2.5 | 2.5 | 2.5 | 2.5 | 2.5 |
| Vitamin B12 (Cyanocobalamin) | 1.0 | 1.0 | 1.0 | 1.0 | 1.0 | 1.0 |

| Active Ingredient | Amount (mg) | | | | | |
|---|---|---|---|---|---|---|
| C₂₆₋₃₄ acid (5n-3) | 120.0 | 120.0 | 120.0 | 120.0 | 120.0 | 120.0 |
| DHA | 120.0 | 120.0 | 120.0 | 120.0 | 120.0 | 120.0 |
| EPA | 120.0 | 120.0 | 120.0 | 120.0 | 120.0 | 120.0 |
| Vitamin C (Ascorbic acid or equivalent) | 500.0 | 500.0 | 500.0 | 500.0 | 500.0 | 500.0 |
| Vitamin E (alpha tocopherol or equivalent) | 180.0 | 180.0 | 180.0 | 180.0 | 180.0 | 180.0 |
| Zinc (Zinc oxide or equivalent) | 80.0 | 80.0 | 80.0 | 25.0 | 25.0 | 25.0 |
| Copper (Copper oxide or equivalent) | 2.0 | 2.0 | 2.0 | 2.0 | 2.0 | 2.0 |
| Lutein (R′R′) | 10.0 | 10.0 | 10.0 | 10.0 | 10.0 | 10.0 |
| Zeaxanthin (R′R′) | 2.0 | 2.0 | 2.0 | 2.0 | 2.0 | 2.0 |
| Vitamin B1 (Thiamin) | 1.5 | 3.0 | 4.5 | 1.5 | 3.0 | 4.5 |
| Vitamin B2 (Riboflavin) | 1.7 | 3.4 | 5.1 | 1.7 | 3.4 | 5.1 |
| Vitamin B3 (Nicotinic acid and/or niacinamide) | 20.0 | 40.0 | 60.0 | 20.0 | 40.0 | 60.0 |
| Vitamin B5 (Pantothenic acid) | 10.0 | 20.0 | 30.0 | 10.0 | 20.0 | 30.0 |
| Vitamin B6 (Pyridoxine) | 50.0 | 50.0 | 50.0 | 50.0 | 50.0 | 50.0 |
| Vitamin B7 (Biotin) | 0.03 | 0.06 | 0.09 | 0.03 | 0.06 | 0.09 |
| Vitamin B9 (Folic acid and/or Methyl tetrahydrofolate) | 2.5 | 2.5 | 2.5 | 2.5 | 2.5 | 2.5 |
| Vitamin B12 (Cyanocobalamin) | 1.0 | 1.0 | 1.0 | 1.0 | 1.0 | 1.0 |

| Active Ingredient | Amount (mg) | | | | | |
|---|---|---|---|---|---|---|
| C₂₆₋₃₄ acid (5n-3) | 6.0 | 6.0 | 6.0 | 6.0 | 6.0 | 6.0 |
| DHA | 150.0 | 150.0 | 150.0 | 150.0 | 150.0 | 150.0 |
| EPA | 100.0 | 100.0 | 100.0 | 100.0 | 100.0 | 100.0 |
| Vitamin C (Ascorbic acid or equivalent) | 500.0 | 500.0 | 500.0 | 500.0 | 500.0 | 500.0 |
| Vitamin E (alpha tocopherol or equivalent) | 180.0 | 180.0 | 180.0 | 180.0 | 180.0 | 180.0 |
| Zinc (Zinc oxide or equivalent) | 80.0 | 80.0 | 80.0 | 25.0 | 25.0 | 25.0 |
| Copper (Copper oxide or equivalent) | 2.0 | 2.0 | 2.0 | 2.0 | 2.0 | 2.0 |
| Lutein (R′R′) | 10.0 | 10.0 | 10.0 | 10.0 | 10.0 | 10.0 |
| Zeaxanthin (R′R′) | 2.0 | 2.0 | 2.0 | 2.0 | 2.0 | 2.0 |
| Vitamin B1 (Thiamin) | 1.5 | 3.0 | 4.5 | 1.5 | 3.0 | 4.5 |
| Vitamin B2 (Riboflavin) | 1.7 | 3.4 | 5.1 | 1.7 | 3.4 | 5.1 |
| Vitamin B3 (Nicotinic acid and/or niacinamide) | 20.0 | 40.0 | 60.0 | 20.0 | 40.0 | 60.0 |
| Vitamin B5 (Pantothenic acid) | 10.0 | 20.0 | 30.0 | 10.0 | 20.0 | 30.0 |
| Vitamin B6 (Pyridoxine) | 50.0 | 50.0 | 50.0 | 50.0 | 50.0 | 50.0 |
| Vitamin B7 (Biotin) | 0.03 | 0.06 | 0.09 | 0.03 | 0.06 | 0.09 |
| Vitamin B9 (Folic acid and/or Methyl tetrahydrofolate) | 2.5 | 2.5 | 2.5 | 2.5 | 2.5 | 2.5 |
| Vitamin B12 (Cyanocobalamin) | 1.0 | 1.0 | 1.0 | 1.0 | 1.0 | 1.0 |

| Active Ingredient | Amount (mg) | | | | | |
|---|---|---|---|---|---|---|
| C₂₆₋₃₄ acid (5n-3) | 2.0 | 2.0 | 2.0 | 2.0 | 2.0 | 2.0 |
| DHA | 100.0 | 100.0 | 100.0 | 100.0 | 100.0 | 100.0 |
| EPA | 0 | 0 | 0 | 0 | 0 | 0 |
| Vitamin C (Ascorbic acid or equivalent) | 500.0 | 500.0 | 500.0 | 500.0 | 500.0 | 500.0 |
| Vitamin E (alpha tocopherol or equivalent) | 180.0 | 180.0 | 180.0 | 180.0 | 180.0 | 180.0 |
| Zinc (Zinc oxide or equivalent) | 80.0 | 80.0 | 80.0 | 25.0 | 25.0 | 25.0 |
| Copper (Copper oxide or equivalent) | 2.0 | 2.0 | 2.0 | 2.0 | 2.0 | 2.0 |
| Lutein (R′R′) | 10.0 | 10.0 | 10.0 | 10.0 | 10.0 | 10.0 |
| Zeaxanthin (R′R′) | 2.0 | 2.0 | 2.0 | 2.0 | 2.0 | 2.0 |
| Vitamin B1 (Thiamin) | 1.5 | 3.0 | 4.5 | 1.5 | 3.0 | 4.5 |
| Vitamin B2 (Riboflavin) | 1.7 | 3.4 | 5.1 | 1.7 | 3.4 | 5.1 |
| Vitamin B3 (Nicotinic acid and/or niacinamide) | 20.0 | 40.0 | 60.0 | 20.0 | 40.0 | 60.0 |
| Vitamin B5 (Pantothenic acid) | 10.0 | 20.0 | 30.0 | 10.0 | 20.0 | 30.0 |
| Vitamin B6 (Pyridoxine) | 50.0 | 50.0 | 50.0 | 50.0 | 50.0 | 50.0 |
| Vitamin B7 (Biotin) | 0.03 | 0.06 | 0.09 | 0.03 | 0.06 | 0.09 |
| Vitamin B9 (Folic acid and/or Methyl tetrahydrofolate) | 2.5 | 2.5 | 2.5 | 2.5 | 2.5 | 2.5 |
| Vitamin B12 (Cyanocobalamin) | 1.0 | 1.0 | 1.0 | 1.0 | 1.0 | 1.0 |

| Active Ingredient | Amount (mg) | | | | | |
|---|---|---|---|---|---|---|
| C₂₆₋₃₄ acid (5n-3) | 0.4 | 0.4 | 0.4 | 0.4 | 0.4 | 0.4 |
| DHA | 0 | 0 | 0 | 0 | 0 | 0 |
| EPA | 0 | 0 | 0 | 0 | 0 | 0 |
| Vitamin C (Ascorbic acid or equivalent) | 500.0 | 500.0 | 500.0 | 500.0 | 500.0 | 500.0 |
| Vitamin E (alpha tocopherol or equivalent) | 180.0 | 180.0 | 180.0 | 180.0 | 180.0 | 180.0 |
| Zinc (Zinc oxide or equivalent) | 80.0 | 80.0 | 80.0 | 25.0 | 25.0 | 25.0 |
| Copper (Copper oxide or equivalent) | 2.0 | 2.0 | 2.0 | 2.0 | 2.0 | 2.0 |
| Lutein (R′R′) | 10.0 | 10.0 | 10.0 | 10.0 | 10.0 | 10.0 |
| Zeaxanthin (R′R′) | 2.0 | 2.0 | 2.0 | 2.0 | 2.0 | 2.0 |
| Vitamin B1 (Thiamin) | 1.5 | 3.0 | 4.5 | 1.5 | 3.0 | 4.5 |
| Vitamin B2 (Riboflavin) | 1.7 | 3.4 | 5.1 | 1.7 | 3.4 | 5.1 |
| Vitamin B3 (Nicotinic acid and/or niacinamide) | 20.0 | 40.0 | 60.0 | 20.0 | 40.0 | 60.0 |
| Vitamin B5 (Pantothenic acid) | 10.0 | 20.0 | 30.0 | 10.0 | 20.0 | 30.0 |
| Vitamin B6 (Pyridoxine) | 50.0 | 50.0 | 50.0 | 50.0 | 50.0 | 50.0 |
| Vitamin B7 (Biotin) | 0.03 | 0.06 | 0.09 | 0.03 | 0.06 | 0.09 |
| Vitamin B9 (Folic acid and/or Methyl tetrahydrofolate) | 2.5 | 2.5 | 2.5 | 2.5 | 2.5 | 2.5 |
| Vitamin B12 (Cyanocobalamin) | 1.0 | 1.0 | 1.0 | 1.0 | 1.0 | 1.0 |

| Active Ingredient | Amount (mg) | | | | | |
|---|---|---|---|---|---|---|
| C₂₆₋₃₄ acid (5n-3) | 1.2 | 1.2 | 1.2 | 1.2 | 1.2 | 1.2 |
| DHA | 0 | 0 | 0 | 0 | 0 | 0 |
| EPA | 0 | 0 | 0 | 0 | 0 | 0 |
| Vitamin C (Ascorbic acid or equivalent) | 500.0 | 500.0 | 500.0 | 500.0 | 500.0 | 500.0 |
| Vitamin E (alpha tocopherol or equivalent) | 180.0 | 180.0 | 180.0 | 180.0 | 180.0 | 180.0 |
| Zinc (Zinc oxide or equivalent) | 80.0 | 80.0 | 80.0 | 25.0 | 25.0 | 25.0 |
| Copper (Copper oxide or equivalent) | 2.0 | 2.0 | 2.0 | 2.0 | 2.0 | 2.0 |
| Lutein (R′R′) | 10.0 | 10.0 | 10.0 | 10.0 | 10.0 | 10.0 |
| Zeaxanthin (R′R′) | 2.0 | 2.0 | 2.0 | 2.0 | 2.0 | 2.0 |
| Vitamin B1 (Thiamin) | 1.5 | 3.0 | 4.5 | 1.5 | 3.0 | 4.5 |
| Vitamin B2 (Riboflavin) | 1.7 | 3.4 | 5.1 | 1.7 | 3.4 | 5.1 |
| Vitamin B3 (Nicotinic acid and/or niacinamide) | 20.0 | 40.0 | 60.0 | 20.0 | 40.0 | 60.0 |
| Vitamin B5 (Pantothenic acid) | 10.0 | 20.0 | 30.0 | 10.0 | 20.0 | 30.0 |
| Vitamin B6 (Pyridoxine) | 50.0 | 50.0 | 50.0 | 50.0 | 50.0 | 50.0 |
| Vitamin B7 (Biotin) | 0.03 | 0.06 | 0.09 | 0.03 | 0.06 | 0.09 |
| Vitamin B9 (Folic acid and/or Methyl tetrahydrofolate) | 2.5 | 2.5 | 2.5 | 2.5 | 2.5 | 2.5 |
| Vitamin B12 (Cyanocobalamin) | 1.0 | 1.0 | 1.0 | 1.0 | 1.0 | 1.0 |

1. A composition for visual health comprising at least one very long chain fatty acid chosen from C₂₆₊ acids (5n-3), prodrugs thereof, and pharmaceutically acceptable salts of any of the foregoing.

2. The composition of claim 1, wherein the composition comprises 1 mg to 1000 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3).

3. The composition of claim 1, wherein the composition comprises 10 mg to 500 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3).

4. The composition of claim 1, wherein the composition comprises 25 mg to 100 mg of the at least one very long chain fatty acid chosen from C₂₆₊ acids (5n-3) or an equivalent amount of a prodrug, pharmaceutically acceptable salt, and/or pharmaceutically acceptable salt of a prodrug of at least one very long chain fatty acid chosen from C₂₆₊ acids (5n-3).

5. The composition of claim 1, wherein the composition comprises 40 mg of at least one very long chain fatty acid chosen from C₂₆₊ acids (5n-3) or an equivalent amount of prodrugs, pharmaceutically acceptable salts, and/or pharmaceutically acceptable salts of prodrugs of C₂₆₊ acid (5n-3) groups.

6. The composition of claim 1, wherein the C₂₆₊ acids (5n-3) is a C₂₆ acid (5n-3), C₂₈ acid (5n-3), C₃₀ acid (5n-3), C₃₂ acid (5n-3), and/or C₃₄ acid (5n-3).

7. The composition of claim 1, wherein the composition further comprises at least one additional component chosen from:
at least one very long chain fatty acid chosen from C₂₆₊ acids (6n-3), prodrugs thereof, pharmaceutically acceptable salts thereof, and pharmaceutically acceptable salt of a prodrug of at least one very long chain fatty acid chosen from C₂₆₊ acids (6n-3).
docosahexanoic acid, prodrugs thereof, and/or pharmaceutically acceptable salts of any of the foregoing;
eicosapentanoic acid, prodrugs thereof, and/or pharmaceutically acceptable salts of any of the foregoing;
vitamin C, prodrugs thereof, and/or pharmaceutically acceptable salts of any of the foregoing;
vitamin E, prodrugs thereof, and/or pharmaceutically acceptable salts of any of the foregoing;
zinc, prodrugs thereof, and/or pharmaceutically acceptable salts of any of the foregoing;
copper, prodrugs thereof, and/or pharmaceutically acceptable salts of any of the foregoing;
lutein, prodrugs thereof, and/or pharmaceutically acceptable salts of any of the foregoing; and
zeaxanthin, prodrugs thereof, and/or pharmaceutically acceptable salts of any of the foregoing.

8. The composition of claim 1, wherein the composition further comprises 20 mg to 350 mg of docosahexanoic acid or an equivalent amount of a prodrug, a pharmaceutically acceptable salt, and/or a pharmaceutically acceptable salt of a prodrug of docosahexanoic acid.

9. The composition of claim 1, wherein the composition further comprises 20 mg to 650 mg of eicosapentanoic acid or an equivalent amount of a prodrug, a pharmaceutically acceptable salt, and/or a pharmaceutically acceptable salt of a prodrug of eicosapentanoic acid.

10. The composition of claim 1, wherein the composition further comprises 350 mg to 800 mg of vitamin C or an equivalent amount of a prodrug, a pharmaceutically acceptable salt, and/or a pharmaceutically acceptable salt of a prodrug of vitamin C.

11. The composition of claim 1, wherein the composition further comprises 50 mg to 550 mg of vitamin E or an equivalent amount of a prodrug, a pharmaceutically acceptable salt, and/or a pharmaceutically acceptable salt of a prodrug of vitamin E.

12. The composition of claim 1, wherein the composition further comprises 20 mg to 90 mg of zinc or an equivalent amount of a prodrug, a pharmaceutically acceptable form, and/or a pharmaceutically acceptable salt of a prodrug of zinc.

13. The composition of claim 1, wherein the composition further comprises 1.5 mg to 2.5 mg of copper or an equivalent amount of a prodrug, a pharmaceutically acceptable form, and/or a pharmaceutically acceptable salt of a prodrug of copper.

14. The composition of claim 1, wherein the composition further comprises 5 mg to 50 mg of lutein or an equivalent amount of a prodrug, a pharmaceutically acceptable salt, and/or a pharmaceutically acceptable salt of a prodrug of lutein.

15. The composition of claim 1, wherein the composition comprises 1 mg to 20 mg of zeaxanthin or an equivalent amount of a prodrug, a pharmaceutically acceptable salt, and/or a pharmaceutically acceptable salt of a prodrug of zeaxanthin.

16. The composition of claim 1, wherein the composition comprises: optionally 1 mg to 200 mg at least one C₂₆₊ acid (6n-3) or an equivalent amount of a prodrug, pharmaceutically acceptable salt, and/or pharmaceutically acceptable salt of a prodrug of at least one C₂₆₊ acid (6n-3); 40 mg at least one C₂₆₊ acid (5n-3) or an equivalent amount of a prodrug, pharmaceutically acceptable salt, and/or pharmaceutically acceptable salt of a prodrug of at least one C₂₆₊ acid (5n-3); 200 mg docosahexanoic acid or an equivalent amount of a prodrug, pharmaceutically acceptable salt, and/or pharmaceutically acceptable salt of a prodrug of docosahexanoic acid; and 40 mg eicosapentanoic acid or an equivalent amount of a prodrug, pharmaceutically acceptable salt, and/or pharmaceutically acceptable salt of a prodrug of eicosapentanoic acid.

17. The composition of claim 1, wherein the composition comprises: optionally 1 mg to 200 mg C₂₆₊ acid (6n-3) or an equivalent amount of a prodrug, pharmaceutically acceptable salt, and/or pharmaceutically acceptable salt of a prodrug of C₂₆₊ acid (6n-3); 40 mg at least one C₂₆₋₃₄ acid (5n-3) or an equivalent amount of a prodrug, pharmaceutically acceptable salt, and/or pharmaceutically acceptable salt of a prodrug of at least one C₂₆₋₃₄ acid (5n-3); 200 mg docosahexanoic acid or an equivalent amount of a prodrug, pharmaceutically acceptable salt, and/or pharmaceutically acceptable salt of a prodrug of docosahexanoic acid; and 40 mg eicosapentanoic acid or an equivalent amount of a prodrug, pharmaceutically acceptable salt, and/or pharmaceutically acceptable salt of a prodrug of eicosapentanoic acid.

18. The composition of claim 1, wherein the composition comprises: 500.0 mg vitamin C or an equivalent amount of a prodrug, pharmaceutically acceptable salt, and/or pharmaceutically acceptable salt of a prodrug of vitamin C; 180.0 mg vitamin E or an equivalent amount of a prodrug, pharmaceutically acceptable salt, and/or pharmaceutically acceptable salt of a prodrug of vitamin E; 80.0 mg zinc or an equivalent amount of a prodrug, pharmaceutically acceptable salt, and/or pharmaceutically acceptable salt of a prodrug of zinc; 2.0 mg copper or an equivalent amount of a prodrug, pharmaceutically acceptable salt, and/or pharmaceutically acceptable salt of a prodrug of zinc; up to 200.0 mg at least one C₂₆₊ acid (6n-3) or an equivalent amount of a prodrug, pharmaceutically acceptable salt, and/or pharmaceutically acceptable salt of a prodrug of at least one C₂₆₊ acid (6n-3); 200.0 mg docosahexanoic acid or an equivalent amount of a prodrug, pharmaceutically acceptable salt, and/or pharmaceutically acceptable salt of a prodrug of docosahexanoic acid; 40.0 mg at least one C₂₆₊ acid (5n-3); and 40.0 mg eicosapentanoic acid or an equivalent amount of a prodrug, pharmaceutically acceptable salt, and/or pharmaceutically acceptable salt of a prodrug of eicosapentanoic acid.

19. The composition of claim 1, wherein the composition comprises: 500.0 mg vitamin C or an equivalent amount of a prodrug, pharmaceutically acceptable salt, and/or pharmaceutically acceptable salt of a prodrug of vitamin C; 180.0 mg vitamin E or an equivalent amount of a prodrug, pharmaceutically acceptable salt, and/or pharmaceutically acceptable salt of a prodrug of vitamin E; 80.0 mg zinc or an equivalent amount of a prodrug, pharmaceutically acceptable salt, and/or pharmaceutically acceptable salt of a prodrug of zinc; 2.0 mg copper or an equivalent amount of a prodrug, pharmaceutically acceptable salt, and/or pharmaceutically acceptable salt of a prodrug of copper; up to 200.0 mg C₂₆₊ acid (6n-3) or an equivalent amount of a prodrug, pharmaceutically acceptable salt, and/or pharmaceutically acceptable salt of a prodrug of C₂₆₊ acid (6n-3); 200 mg docosahexanoic acid or an equivalent amount of a prodrug, pharmaceutically acceptable salt, and/or pharmaceutically acceptable salt of a prodrug of docosahexanoic acid; 40.0 mg at least one C₂₆₋₃₄ acid (5n-3) or an equivalent amount of a prodrug, pharmaceutically acceptable salt, and/or pharmaceutically acceptable salt of a prodrug of C₂₆₋₃₄ acid (5n-3); and 40.0 mg eicosapentanoic acid or an equivalent amount of a prodrug, pharmaceutically acceptable salt, and/or pharmaceutically acceptable salt of a prodrug of eicosapentanoic acid.

20. The composition of claim 18, further comprising 10.0 mg lutein or an equivalent amount of a prodrug, pharmaceutically acceptable salt, and/or pharmaceutically acceptable salt of a prodrug of lutein and 2.0 mg zeaxanthin or an equivalent amount of a prodrug, pharmaceutically acceptable salt, and/or pharmaceutically acceptable salt of a prodrug of zeaxanthin.

21. The composition of claim 1, further comprising at least one additional active ingredient.

22. The composition of claim 21, wherein the at least one additional active ingredient is chosen from
Vitamin C, prodrugs thereof, and pharmaceutically acceptable salts of any of the foregoing;
Vitamin E, prodrugs thereof, and pharmaceutically acceptable salts of any of the foregoing;
zinc, prodrugs thereof, and pharmaceutically acceptable salts of any of the foregoing;
copper, prodrugs thereof, and pharmaceutically acceptable salts of any of the foregoing;
Vitamin B1, prodrugs thereof, and pharmaceutically acceptable salts of any of the foregoing;
Vitamin B2, prodrugs thereof, and pharmaceutically acceptable salts of any of the foregoing;
Vitamin B3, prodrugs thereof, and pharmaceutically acceptable salts of any of the foregoing;
Vitamin B5, prodrugs thereof, and pharmaceutically acceptable salts of any of the foregoing;
Vitamin B6, prodrugs thereof, and pharmaceutically acceptable salts of any of the foregoing;
Vitamin B7, prodrugs thereof, and pharmaceutically acceptable salts of any of the foregoing;
Vitamin B9, prodrugs thereof, and pharmaceutically acceptable salts of any of the foregoing;
Vitamin B12, prodrugs thereof, and pharmaceutically acceptable salts of any of the foregoing;
lutein, prodrugs thereof, and pharmaceutically acceptable salts of any of the foregoing; and
zeaxanthin, prodrugs thereof, and pharmaceutically acceptable salts of any of the foregoing.

23. The composition of claim 1, wherein the composition is in the form of one or more tablets, capsules, soft gels, liquid, or powder.

24. The composition of claim 1, wherein the composition is a daily dosage.

25. A method of delaying the progression and/or onset macular degeneration, the method comprising administering to a subject in need thereof a composition according to claim 1.

26. A method for treating and/or preventing at least one disease, disorder, and/or condition associated with a loss of visual acuity, the method comprising administering to a subject a composition according to claim 1.

27. The method of claim 25, wherein the composition is administered daily to the subject in one, two, three, or four daily doses.
